SEQUENCE LISTING (7 sequences)

SEQ ID NO: 1 - protein, 623 aa, Adeno-associated virus 1
MPGFYEIVIKVPSDLDEHLPGISDSFVSWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYEPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 2 - protein, 623 aa, Adeno-associated virus 6
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 3 - protein, 624 aa, Adeno-associated virus 3
MPGFYEIVLKVPSDLDEHLPGISNSFVNWVAEKEWELPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTKQEVKDFFRWASDHVTDVAHEFYVRKGGAKKRPASNDADVSEPKRQCTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 4 - protein, 624 aa, Adeno-associated virus 3B; identical to SEQ ID NO: 3

SEQ ID NO: 5 - protein, 623 aa, Adeno-associated virus 4
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 6 - protein, 621 aa, Adeno-associated virus 2
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 7 - protein, 610 aa, Adeno-associated virus 5
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

(19) United States
(12) Patent Application Publication
(10) Pub. No.: US 2003/0224404 A1
(43) Pub. Date: Dec. 4, 2003

(54) High throughput directed evolution of nucleic acids by rational mutagenesis

(76) Inventors: Manuel Vega, Vigneux-sur-Seine (FR); Lila Drittanti, Vigneux-sur-Seine (FR)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/375,192

(22) Filed: Feb. 24, 2003

Related U.S. Application Data
(60) Provisional application No. 60/360,085, filed on Feb. 25, 2002.

(51) Int. Cl. C12Q 1/68; C07H 21/02; C07H 21/04; C12P 19/34; C12P 21/02; C12N 5/06

(52) U.S. Cl. 435/6; 435/69.1; 435/320.1; 435/91.2; 435/325; 530/350; 536/23.1

(57) ABSTRACT

Method and systems for the high throughput directed evolution of functional nucleic acid molecules, such as cis-acting elements, particularly those that act in complex biological settings, are provided.

### RELATED APPLICATIONS

Benefit of priority under 35 U.S.C. §119(e) to U.S. provisional application Serial No. 60/360,085, filed Feb. 25, 2002, to Manuel Vega and Lila Drittanti entitled “HIGH THROUGHPUT DIRECTED EVOLUTION OF NUCLEIC ACIDS BY RATIONAL MUTAGENESIS” is claimed. This application also is related to copending U.S. application Ser. No. 10/022,249, filed Dec. 17, 2001, to Manuel Vega and Lila Drittanti entitled “HIGH THROUGHPUT DIRECTED EVOLUTION BY RATIONAL MUTAGENESIS” and to U.S. provisional application Serial No. 60/315,382, filed Aug. 27, 2001, to Manuel Vega, Lila Drittanti and Marjorie Flaux, entitled “HIGH THROUGHPUT DIRECTED EVOLUTION BY RATIONAL MUTAGENESIS.”

The subject matter of each of these applications and provisional application is incorporated in its entirety by reference thereto.

### FIELD OF THE INVENTION

Processes and systems for the high throughput directed evolution of nucleic acid molecules, peptides and proteins, particularly those that act in complex biological settings, are provided. The nucleic acid molecules include cis acting sequences of nucleotides and those responsive to trans-acting factors, and include, promoters, enhancers, silencers; sequences responsive to trans acting factors, such as nucleic acid binding proteins, transcription activating factors, scaffold attachment sites; RNA, antisense molecules and other nucleic acid molecules. The proteins and peptides include, but are not limited to, intracellular proteins, messenger/signaling/hormone proteins and viral proteins.

### BACKGROUND

Directed evolution refers to biotechnological processes for optimizing the activity of proteins by means of random changes introduced into selected respective genes. Directed evolution involves the creation of a library of mutated genes, and then selection of the mutants that encode proteins having desired properties. The process can be an iterative one in which gene products that have improvement in a desired property are subjected to further cycles of mutation and screening. Directed evolution provides a way to adapt natural proteins to work in new chemical or biological environments, and/or to elicit new functions. The potential plasticity of proteins is such that chances exist that for every new challenge, such as a new environment and desired new or altered activity, it should be possible, given a sufficient pool of modified proteins (or encoding nucleic acids), that an appropriately ‘evolved’ protein could be found that would have a desired activity. The problem is in generating and then identifying the appropriate sequence.

There have been practical approaches to this problem (see, e.g., U.S. Pat. Nos. 6,096,548; 6,117,679; 6,165,793; 6,180,406; 6,132,970; 6,171,820; 6,238,884; 6,174,673; 6,057,103; 6,001,574; 5,763,239; 5,837,500; 5,571,698; 6,156,509; 5,723,323; 5,862,514; 5,871,974; 5,779,434 and others). Typically theses approaches are of two types. One is-a purely “rational” approach that is based on the assumption that the optimized proteins can be rationally designed. This, however, requires sufficient information regarding the laws that govern protein folding, molecular interactions, intra-molecular forces and other dynamics of protein activity. This rational approach is extremely dependent on a number of variables and parameters that are not known. Consequently, although useful in some specific cases and applications, the rational approach intended to ‘predict’ protein structure remains limited in applicability.

In contrast to the rational approach, random approaches have also been employed. One random approach requires synthesis of all possible protein sequences or a statistically sufficient large number of proteins and then screening them to identify proteins having the desired activity or property. Since the resources to synthesize all possible theoretical sequences of a single protein is not possible, this approach is impracticable. Other random approaches are based on gene shuffling methods, which are PCR-based methods that generate random rearrangements between two or more sequence-related genes to randomly generate variants of the gene.

The development and scope of directed evolution, thus, has been limited, and its potential remains to be exploited. In order to exploit the potential of directed evolution, alternative approaches for generating and identifying evolved proteins are needed. It is an object herein to provide methods and products to exploit the potential of directed evolution.

### SUMMARY

Provided herein are methods for performing directed evolution for the optimization of proteins and nucleic acid molecules that function in complex biological settings. Methods of high throughput directed evolution of proteins and nucleic acids are provided. In practicing the methods, each molecule is individually designed, produced, processed, screened and tested in a high throughput format. In the overall process, neither random or combinatorial methods nor mixtures of molecules are used; such methods optionally can be used in certain of the steps.

Methods for production of molecules, particularly nucleic acid molecules, having a predetermined property are provided. The methods include the steps of identifying a region of a targeted nucleic acid molecule for modification; producing a population of sets of nucleic acid molecules that include a modified target sequence of nucleotides; introducing each set of nucleic acid molecules into host cells and expressing a protein whose expression is regulated by the target functional nucleic acid; and individually screening the sets of encoded proteins or expression thereof to identify the target functional nucleic acid molecules whose activity is altered. Each target functional nucleic acid that modifies or results in a change in expression of a protein or other cellular molecule or activity is designated a hit.

In other embodiments, the steps for production of functional molecules nucleic acid molecule having a predetermined property or properties, includes the steps of producing a population of sets of modified nucleic acid molecules that encode modified forms of a target functional nucleic acid, where each modified functional nucleic acid molecule is operably associated with a nucleic acid region encoding a reporter, such as a detectable protein; introducing each set of nucleic acid molecules into host cells under conditions that express a reporter when using a wild-type functional nucleic acid region; individually screening the sets of nucleic acid molecules encoding reporter proteins to identify one or more target modified functional nucleic acid regions that has activity that differs from the unmodified functional nucleic acid molecule. Reporters include, but are not limited to, detectable proteins, such as fluorescent proteins and enzymes, and detectable nucleic acids.

In all embodiments, an exemplary desired activity is an increased (or decreased) production of a protein encoded by a sequence of nucleotides operatively linked to coding region of the protein. In all embodiments, the host cells are present in an addressable collection, such as a positionally addressable array or linked to encoded particles. Addressable arrays include solid supports with positionally addressable loci, such as wells. In such arrays, each locus contains one set of cells. Modified target nucleic acid molecules can be produced by any suitable method, including, but not limited to, systematically changing each base in the target protein to a pre-selected base, and other methods, such as, for example, nucleic acid shuffling, recombination, site-directed mutagenesis and random mutagenesis.

Each variant is introduced into a host and the resulting cells are maintained separately, such as in an addressable array of wells in a microtiter plate or other substrate with discrete locations for performing reactions or retaining molecules of interest. Typical formats are 96 loci, and multiples thereof (384, 1536, 3072, . . . 96×n, where n is 1 to any number desired, such as 10, 20, 30, 50 . . . 100), although any convenient number of loci can be employed.

In all embodiments, identified molecules with altered activity are designated hits. Target molecules include prokaryotic and eukaryotic genes and vectors, such as viral vectors and plasmids. Exemplary viral vectors include, but are not limited to, vectors based on or derived herpes simplex virus (HSV), pseudorabies virus and other herpes viruses, MuMLV, MoMLV, feline leukemia virus, and HIV and other lentiviruses, such as, for example, vectors derived from adenoviruses, adeno-associated viruses, vaccinia viruses and retroviruses. The host cells can be prokaryotic cells or eukaryotic cells that are transduced with the vectors. After introduction of a viral vector containing the modified sequence of nucleotides, the titer of the viral vectors in each set of cells can be assess.

In all embodiments, the sequence of nucleic acids, such as a promoter, can be gradually converted by mutagenesis into corresponding consensus sequences known to have higher activity by accumulation of single point mutations from the borders towards the middle of the consensus sequences to generate a collection of mutants, where each mutant differs from the preceding mutant in that a new mutation that is added. In other embodiments, the modifications are single base deletion mutants generated such that each mutant either increases or decreases the distance between one or more bases in a consensus sequence, such as in a promoter, by virtue of insertions or deletions. In all embodiments, the modifications can be effected in a selected portion of a target functional nucleic acid molecule or throughout the molecule. The change in assessed activity can be at least about 0.1%, 0.5%, 1%, 5%, 10%, 20%, 30%, 40%, 50%, 60%, 75%, 80%, 90%, 100%, 200%, 500%, 1000% or greater. The change can be manifested as an increase, decrease or other alteration of an activity.

Target functional nucleic acid molecules, include, but are not limited to, cis acting regulatory sequences, antisense nucleic acid and RNA, including dsRNA. These include, for example promoters, enhancers, silencers, protein binding domains, ribosomal RNA and ribozymes and tRNA, interfering RNA (RNAi) and antisense molecules. Target functional nucleic acid molecules include sequences of nucleic acids that are modified as described herein. The resulting modified molecules are screened as described herein to identify those having a desired activity.

Molecules in which hits have a desired activity, such as changing of a constitutive promoter into a regulatable promoter, are designated LEADS, and molecules in which leads are combined to produce molecules with the desired predetermined property are designated SUPERLEADS. Thus, each of the methods can further include the steps of modifying the nucleic acid molecules that comprise the hits, to produce a set of modified hits; introducing the each set of modified hits into cells; and individually screening the sets cells that comprise the modified hits to identify one or more cells that contain a candidate functional nucleic acid that has activity that differs from the target functional nucleic acid and has properties that differ from the original hits. The resulting functional nucleic acids are each designated a lead. The modified hits can be produced by any method known to those of skill in the art include, but not limited to, systematically changing each base in the target protein to a pre-selected base, and other methods, such as, for example, nucleic acid shuffling, recombination, site-directed mutagenesis and random mutagenesis. Thus, leads can be identified by preparing modified hits that are produced by systematically replacing base that is a hit, with each of the remaining two bases, to produce nucleic acid molecules each differing by at least one base.

The resulting leads can be introduced into cells and the cells screened to identify nucleic acid molecules that are optimized leads. Two, three and up to all of the nucleic acid leads can be recombined. Recombining can be effected by any suitable method, including but are not limited to, nucleic acid shuffling, recombination, site-directed, random mutagenesis and de novo synthesis.

As noted, since the process is conducted in a high throughput format, for many embodiments, it is often important to assess the relative numbers of transformed, transduced or transfected cells. Hence the relative (or actual) titer of the vector, such as the recombinant viral vector, must be known to permit analysis of results. For high throughput formats, it is important to assess the relative or actual concentration of the viral vector (or plasmid) so that results can be compared among all cells and variants. Methods for titering (determining the concentration) of the nucleic acid encoding the variant and/or the recombinant virus are also provided.

The methods require accurate titering of the viruses in a collection or among collections (libraries) so that the activities of the screened mutant proteins can be compared. Provided are general methods for the quantitative assessment of the parameters of activity corresponding to the individual variants in the library, based upon intracellular serial dilution generated by precise titering with the gene transfer viral vectors Any method permits accurate titering may be used, including that described International PCT application No. PCT/FR01/01366, based on French application no. 0005852, filed May 9, 2000, and published as International PCT application No. WO 01/186291. A method of titering, designated Tagged Replication and Expression Enhancement Technology (TREE™; see, EP1244912) is provided herein.

Each of the different cells is separately screened by a suitable assay, and the results analyzed. Methods for assessing the interactions in biological systems, such as a Hill-based analysis (see, published International PCT application No. WO 01/44809 based on PCT No. PCT/FR00/03503, December, 2000, and the description herein), or a second order polynomial or other algorithm that describes the interaction between cells and biological agents to select variants that have a desired property are employed in the methods herein.

Reporter cells are infected with the titered viruses that encode the mutant genes. The mutant genes are expressed and read-out data from either biochemical or cell-based assays, while isolating each mutant/virus physically from the others (i.e. one-by-one analysis), are collected and analyzed. Serial dilution assays (i.e. a series of dilutions for each individual mutant/virus in the library) are used and the biochemical/cell-based assays are performed on each single dilution for each individual mutant/virus. Analysis of the serial dilution readout-data can be performed using any method of analysis that permits one-by-one comparisons. Hill-based analysis (see, published International PCT application No. WO 01/44809 based on PCT No. PCT/FR00/03503, December, 2000, and the description herein) are employed for analysis of the data.

The above methods are used for alteration of nucleic acid molecules or sequences therein, such as functional domains, including but are not limited to, cis acting regulatory regions, RNA, and antisense molecules. For directed evolution of nucleic acid molecules, the methods provided herein include the steps of identifying a nucleic acid molecule or region in a nucleic acid molecule of interest; and creating variants using the methods herein. For example, a nucleic acid molecule or portion thereof, such as a promoter region is identified, such as by obtaining a protein encoding nucleic acid and linking it to a region that contains the transcriptional regulatory sequences, such as at least about 2500 nucleotides, 1000 nucleotides, 500 nucleotides, 250 nucleotides, 100 nucleotides or less upstream of a protein-encoding start site. The transcriptional regulatory sequence can be linked to its native protein-encoding nucleic acid or to a heterologous nucleic acid. In a first step, each nucleotide is systematically changed to another, such that each base AT base pair is changed to, a GC base pair, and each GC base pair is changed to an AT base pair, and then the expression of the linked encoded protein is monitored. Alternatively, each base is changed to another base, and this is optionally repeated with another base, such as G, or with two or three more bases, in order to observe the effects of changing each nucleotide. Any nucleotides whose change alters activity are identified and denoted hits. Each hit position is replaced with each of the remaining three bases and leads are identified. In an optional third screening step, combinations of two or more of the lead positions are altered to ultimately identify modified sequences that have a desired altered activity; these are designated SUPERLEADS. In an exemplary embodiment, the method is applied to promoter regions. Other promoter and cis-acting regulatory sequence optimization approaches also are provided.

Thus provided are methods for the production of a functional nucleic molecule, having a predetermined property including the steps of:

(a) producing a population of sets of nucleic acid molecules that include a target modified functional nucleic acid sequences of nucleic acids in the molecule;

(b) introducing each set of nucleic acid molecules into host cells and expressing a protein whose expression is regulated by the target functional nucleic acid molecule, where the host cells are present in an addressable collection, such as an array; and

(c) individually screening the sets of encoded proteins to identify the target functional nucleic acid molecules whose activity is altered. Each such target functional nucleic acid is designated a hit.

Target functional nucleic acid molecules include cis acting regulatory sequences, antisense nucleic acids and RNA. These include, but are not limited to, promoters, enhancers, silencers, protein binding domains, ribosomal RNA and ribozymes and tRNA and antisense molecules.

In the method for the production of a functional nucleic acid molecule having a predetermined property, each modified functional nucleic acid molecule can be operably associated with a nucleic acid region encoding a reporter, such as a detectable protein or a detectable or identifiable nucleic acid molecule. Each set of nucleic acid molecules into is introduced host cells under conditions that express an encoded reporter protein when using a wild-type functional nucleic acid region. The host cells are present in an addressable collection. The sets of nucleic acid molecules encoding reporters are individually screened to identify one or more target modified functional nucleic acid regions that has activity that differs from the unmodified functional nucleic acid molecule. Each such molecule is designated a hit.

In these methods, each target modified functional nucleic acid molecule is modified to differ by a single base or base pair from the target molecule.

The collections can be addressable arrays, such as a solid support with wells, where each well contains one set of cells.

The nucleic acid molecules can be in viral vectors or bacterial plasmids; and the cells can be eukaryotic cells that are transduced with the vectors or bacterial cells containing the plasmids. The vectors include, but are not limited to, vectors derived from herpes simplex virus (HSV), pseudorabies virus and other herpes virus vectors, MuMLV, MoMLV, feline leukemia virus, and HIV and other lentiviruses, adenoviruses, adeno-associated viruses, herpes viruses, vaccinia viruses and retroviruses.

After identification of the hits, the nucleic acid molecules that comprise the hits are modified to produce a set of modified hits. The modified hits are produced by changing each hit to each of the other nucleic acid bases. These functional nucleic acids are screened to have a selected property. Molecules having such properties are leads.

The modified target functional nucleic acid molecules can be produced by a systematically changing each base in the target protein to a pre-selected base.

The method can further include steps of combining the nucleic acid molecules that are leads to produce molecules with two or more differences from the original molecule. Such molecules are screened for a predetermined property. Identified molecules are designated optimized leads or SUPERLEADS.

Changes in the sequence of the target functional nucleic acid molecule can be effected by systematically changing each base, or converting a consensus sequence to one known to have higher activity by accumulating a series of single point mutations from the borders towards the middle of the consensus sequence to generate a collection of mutants, wherein each mutant differs from the preceding mutant in that a new mutation is added, or by altering the distance between bases that comprise a consensus sequence.

Systems and computer controlled systems for performing the high throughput methods are also provided.

### DESCRIPTION OF THE FIGURES

FIG. 1 summarize various exemplary embodiments of the high throughput processes provided herein. FIG. 1A depicts an embodiment of the process in which an amino acid scan is employed to generate a library of mutants, which are then introduced into viral vectors, such as an adeno-associated viral vector (AAV), a herpes virus, such as herpes simplex virus (HSV) and other herpes virus vectors, a vaccinia virus vector, retroviral vectors, such as MuMLV, MoMLV, feline leukemia virus, and HIV and other lentiviruses, adenovirus vectors and other suitable viral vector, each member of the library is individually tested and phenotypically characterized to identify HITS. FIG. 1B summarizes round 2 in which LEADS are developed by mutagenesis at and/or surrounding the positions identified as HITS; FIG. 1C summarizes the optional next round in which recombination among LEADS is performed to further optimize the LEADS; FIG. 1D depicts the process in mammalian cells; and FIG. 1E depicts the process in bacterial cells.

FIG. 2A depicts an exemplary titering process (in this instance the TREE™ for titering AAV) in a 96 well format; FIG. 2B shows the results and analysis of a titering process performed using the TREE™ procedure; and FIG. 2C shows an exemplary calibration curve for the calculation of the titer using the TREE™ method.

FIGS. 3A and 3B depict “HITS” and “LEADS” respectively for identification of AAV rep mutants “evolved” for increased activity.

FIG. 4 shows the genetic map of AAV, including the location of promoters, and transcripts; amino acid 1 of the Rep 78 gene is at nucleotide 321 in the AAV-2 genome.

FIGS. 5A and 5B show the alignment of amino acid sequences of Rep78 among AAV-1; AAV-6; AAV-3; AAV-3B; AAV-4; AAV-2; AAV-5 sequences, respectively; the hit positions with 100 percent homology among the serotypes are bolded italics, where the position is different (compared to AAV-2, no. 6 in the Figure) in a particular serotype, it is in bold; a sequence indicating relative conservation of sequences among the serotypes is labeled “C”.

Legend:

1 is AAV-1; 2 is AAV-6, 3 is AAV-3, 4 is AAV-3B, 5 is AAV-4, 6 is AAV-2, and 7 is AAV-5;

“.” where the amino acid is present ≧20%;

“:” where the amino acid is present ≧40%;

“+” where the amino acid is present ≧60%;

“*” where the amino acid is present ≧80%; and

where the amino acid is the same amongst all serotypes depicted it is represented by its single letter code.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases.

As used herein, directed evolution refers to methods that adapt natural proteins or protein domains to work in new chemical or biological environments and/or to elicit new functions. It is more a more broad-based technology than DNA shuffling.

As used herein, high-throughput screening (HTS) refers to processes that test a large number of samples, such as samples of test proteins or cells containing nucleic acids encoding the proteins of interest to identify structures of interest or to identify test compounds that interact with the variant proteins or cells containing them. HTS operations are amenable to automation and are typically computerized to handle sample preparation, assay procedures and the subsequent processing of large volumes of data.

As used herein, DNA shuffling is a PCR-based technology that produces random rearrangements between two or more sequence-related genes to generate related, although different, variants of a given gene.

As used herein, “hits” are mutant proteins that have an alteration in any attribute, chemical, physical or biological property in which such alteration is sought. In the methods herein, hits are generally generated by systematically replacing each amino acid in a protein or a domain thereof with a selected amino acid, typically Alanine, Glycine, Serine or any amino acid, as long as each residue is replaced with the same residue. Hits may be generated by other methods known to those of skill in the art and tested by the high throughput methods herein. For purposes herein a Hit typically has activity with respect to the function of interest that differs by at least 10%, 20%, 30% or more from the wild type or native protein. The desired alteration, which is generally a reduction in activity, depends upon the function or property of interest. As used herein, “leads” are “hits” whose activity has been optimized for the particular attribute, chemical, physical or biological property. In the methods herein, leads are generally produced by systematically replacing the hit loci with all remaining 18 amino acids, and identifying those among the resulting proteins that have a desired activity. The leads may be further optimized by replacement of a plurality of “hit” residues. Leads may be generated by other methods known to those of skill in the art and tested by the high throughput methods herein. For purposes herein a lead typically has activity with respect to the function of interest that differs from the native activity, by a desired amount and is at by at least 10%, 20%, 30% or more from the wild type or native protein. Generally a Lead has activity that is 2 to 10 or more times the native protein for the activity of interest. As with hits, the change in the activity is dependent upon the activity that is “evolved.” The desired alteration depends upon the function or property of interest.

As used herein, a functional nucleic acid molecule is a nucleic acid molecule contemplated for rational directed evolution; it generally is a nucleic acid molecule that itself has functional activity other than (or in addition to), encoding a protein. Exemplary functional nucleic acid molecules include, but are not limited to, cis acting regulatory sequences, such as promoters, enhancers, silencers, protein binding domains, and other such sequences, and antisense nucleic acid and RNA, such as ribosomal RNA and ribozymes, and tRNA.

As used herein, MOI is multiplicity of infection.

As used herein, ip, with reference to a virus or recombinant vector, refers to a titer of infectious particles.

As used herein, pp refers to the total number of vector (or virus) physical particles

As used herein, biological and pharmacological activity includes any activity of a biological pharmaceutical agent and includes, but is not limited to, biological efficiency, transduction efficiency, gene/transgene expression, differential gene expression and induction activity, titer, progeny productivity, toxicity, cytotoxicity, immunogenicity, cell proliferation and/or differentiation activity, anti-viral activity, morphogenetic activity, teratogenetic activity, pathogenetic activity, therapeutic activity, tumor suppressor activity, ontogenetic activity, oncogenetic activity, enzymatic activity, pharmacological activity, cell/tissue tropism and delivery.

As used herein, “output signal” refers to parameters that can be followed over time and, if desired, quantified. For example, when a virus infects a cell, the infected cell undergoes a number of changes. Any such change that can be monitored and used to assess infection, is an “output signal,” and the cell is referred to as a “reporter cell.” Output signals include, but are not limited to, enzyme activity, fluorescence, luminescence, amount of product produced and other such signals. Output signals include expression of a viral gene or viral gene product, including heterologous genes (transgenes) inserted into the virus. Such expression is a function of time (“t”) after infection, which in turn is related to the amount of virus used to infect the cell, and, hence, the concentration of virus (“s”) in the infecting composition. For higher concentrations the output signal is higher. For any particular concentration, the output signal increases as a function of time until a plateau is reached. Output signals may also measure the interaction between cells, expressing heterologous genes, and biological agents As used herein, adeno-associated virus (AAV) is a defective and non-pathogenic parvovirus that requires co-infection with either adenovirus or herpes virus for its growth and multiplication, able of providing helper functions. A variety of serotypes are known, and contemplated herein. Such serotypes include, but are not limited to: AAV-1 (Genbank accession no. NC_{—}002077; accession no. VR-645); AAV-2 (Genbank accession no. NC_{—}001401; accession no. VR-680); AAV-3 (Genbank accession no. NC_{—}001729; accession no. VR-681); AAV-3b (Genbank accession no. NC_{—}001863); AAV-4 (Genbank accession no. NC_{—}001829; ATCC accession no. VR-646); AAV-6 (Genbank accession no. NC_{—}001862); and avian associated adeno-virus (ATCC accession no. VR-1449). The preparation and use of AAVs as vectors for gene expression in vitro and for in vivo use for gene therapy is well known (see, e.g., U.S. Pat. Nos. 4,797,368, 5,139,941, 5,798,390 and 6,127,175; Tessier et al. (2001) J. Virol. 75:375-383; Salvetti et al. (1998) Hum Gene Ther 20:695-706; Chadeuf et al. (2000) J Gene Med 2:260-268).

As used herein, the activity of a Rep protein or of a capsid protein refers to any biological activity that can be assessed. In particular, herein, the activity assessed for the rep proteins is the amount (i.e., titer) of AAV produced by a cell.

As used herein, the Hill equation is a mathematical model that relates the concentration of a drug (i.e., test compound or substance) to the response being measured

$$y = \frac{{{y}_{max} [ D ]}^{x}}{{[ D ]}^{n} + {[ {D}_{50} ]}^{n}}$$

where y is the variable being measured, such as a response, signal, yₘₐₓ is the maximal response achievable, [D] is the molar concentration of a drug, [D₅₀] is the concentration that produces a 50% maximal response to the drug, n is the slope parameter, which is 1 if the drug binds to a single site and with no cooperativity between or among sites. A Hill plot is log₁₀ of the ratio of ligand-occupied receptor to free receptor vs. log [D] (M). The slope is n, where a slope of greater than 1 indicates cooperativity among binding sites, and a slope of less than 1 can indicate heterogeneity of binding. This general equation has been employed for assessing interactions in complex biological systems (see, published International PCT application No. WO 01/44809 based on PCT No. PCT/FR00/03503, see, also, EXAMPLES).

As used herein, in the Hill-based analysis (published International PCT application No. WO 01/44809 based on PCT No. PCT/FR00/03503), the parameters, π,κ,τ,ε,η,θ, are as follows:

π potency of the biological agent acting on the assay (cell-based) system;

κ constant of resistance of the assay system to elicit a response to a biological agent;

ε is global efficiency of the process or reaction triggered by the biological agent on the assay system;

τ is the apparent titer of the biological agent;

θ is the absolute titer of the biological agent; and

η is the heterogeneity of the biological process or reaction.

In particular, as used herein, the parameters π (potency) or κ (constant of resistance) are used to respectively assess the potency of a test agent to produce a response in an assay system and the resistance of the assay system to respond to the agent.

As used herein, ε(efficiency), is the slope at the inflection point of the Hill curve (or, in general, of any other sigmoidal or linear approximation), to asses the efficiency of the global reaction (the biological agent and the assay system taken together) to elicit the biological or pharmacological response.

As used herein, τ (apparent titer) is used to measure the limiting dilution or the apparent titer of the biological agent.

As used herein, θ (absolute titer), is used to measure the absolute limiting dilution or titer of the biological agent.

As used herein, η (heterogeneity) measures the existence of discontinuous phases along the global reaction, which is reflected by an abrupt change in the value of the Hill coefficient or in the constant of resistance.

As used herein, a library of mutants refers to a collection of plasmids or other vehicles that carrying (encoding) the gene variants, such that individual plasmid or other vehicles carry individual gene variants. When a library of proteins is contemplated, it will be so-stated.

As used herein, a “reporter cell” is the cell that “reports”, i.e., undergoes the change, in response to introduction of the nucleic acid infection and, therefore, it is named here a reporter cell.

As used herein, “reporter” or “reporter moiety” refers to any moiety that allows for the detection of a molecule of interest, such as a protein expressed by a cell. Typical reporter moieties include, for example, fluorescent proteins, such as red, blue and green fluorescent proteins. For expression in cells, nucleic acid encoding the reporter moiety can be expressed as a fusion protein with a protein of interest or under to the control of a promoter of interest.

As used herein, a titering virus increases or decreases the output signal from a reporter virus, which is a virus that can be detected, such as by a detectable label or signal.

As used herein, phenotype refers to the physical or other manifestation of a genotype (a sequence of a gene). In the methods herein, phenotypes that result from alteration of a genotype are assessed.

As used herein, activity refers to the function or property to be evolved. An active site refers to a site(s) responsible or that participates in conferring the activity or function. The activity or active site evolved (the function or property and the site conferring or participating in conferring the activity) may have nothing to do with natural activities of a protein. For example, it could be an ‘active site’ for conferring immunogenicity (immunogenic sites or epitopes) on a protein.

As used herein, the amino acids, which occur in the various amino acid sequences appearing herein, are identified according to their known, three-letter or one-letter abbreviations (see, Table 1). The nucleotides, which occur in the various nucleic acid fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are presumed to be in the “L” isomeric form. Residues in the “D” isomeric form, which are so-designated, can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in the following Table:

TABLE 1
Table of Correspondence

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions are preferably made in accordance with those set forth in TABLE 2 as follows:

TABLE 2

| Original residue | | Conservative substitution |
|---|---|---|
| Ala | (A) | Gly; Ser |
| Arg | (R) | Lys |
| Asn | (N) | Gln; His |
| Cys | (C) | Ser |
| Gln | (Q) | Asn |
| Glu | (E) | Asp |
| Gly | (G) | Ala; Pro |
| His | (H) | Asn; Gln |
| Ile | (I) | Leu; Val |
| Leu | (L) | Ile; Val |
| Lys | (K) | Arg; Gln; Glu |
| Met | (M) | Leu; Tyr; Ile |
| Phe | (F) | Met; Leu; Tyr |
| Pro | (P) | Ala; Gly |
| Ser | (S) | Thr |
| Thr | (T) | Ser |
| Trp | (W) | Tyr |
| Tyr | (Y) | Trp; Phe |
| Val | (V) | Ile; Leu |

Other substitutions are also permissible and may be determined empirically or in accord with known conservative substitutions.

As used herein, nucleic acids include DNA, RNA and analogs thereof, including protein nucleic acids (PNA) and mixture thereof. Nucleic acids can be single or double stranded. When referring to probes or primers, optionally labeled, with a detectable label, such as a fluorescent or radiolabel, single-stranded molecules are contemplated. Such molecules are typically of a length such that they are statistically unique of low copy number (typically less than 5, preferably less than 3) for probing or priming a library. Generally a probe or primer contains at least 14, 16 or 30 contiguous nucleotides complementary to or identical to a gene of interest. Probes and primers can be 10, 14, 16, 20, 30, 50, 100 or more nucleic acid bases long.

As used herein, by homologous means about greater than 25% nucleic acid sequence identity, preferably 25% 40%, 60%, 80%, 90% or 95%. The intended percentage will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

As used herein, a nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unitary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are, for example, at least 80%, 85%, 90%, 95%, 96%, 97%, 98% or 99%, “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)), and CLUSTALW. For sequences displaying a relatively high degree of homology, alignment can be effected manually by simply lining up the sequences and manually or visually matching the conserved portions.

Therefore, as used herein, the term “identity” represents a comparison between a test and a reference polypeptide or polynucleotide. For example, a test polypeptide may be defined as any polypeptide that is 90% or more identical to a reference polypeptide.

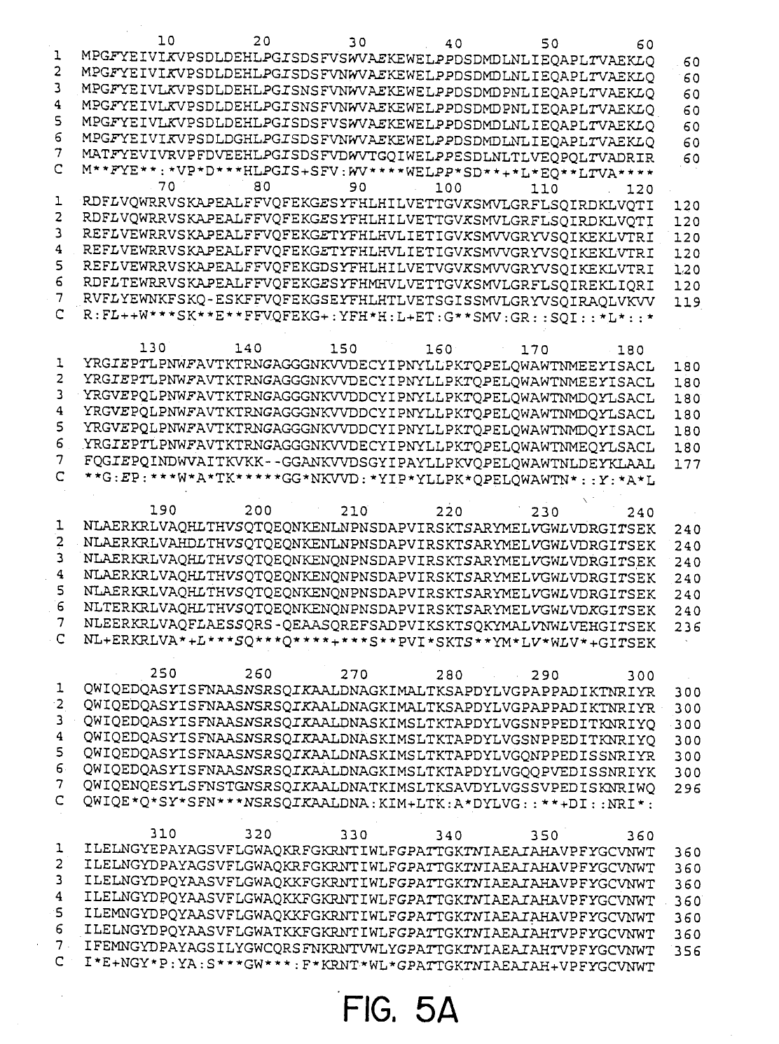

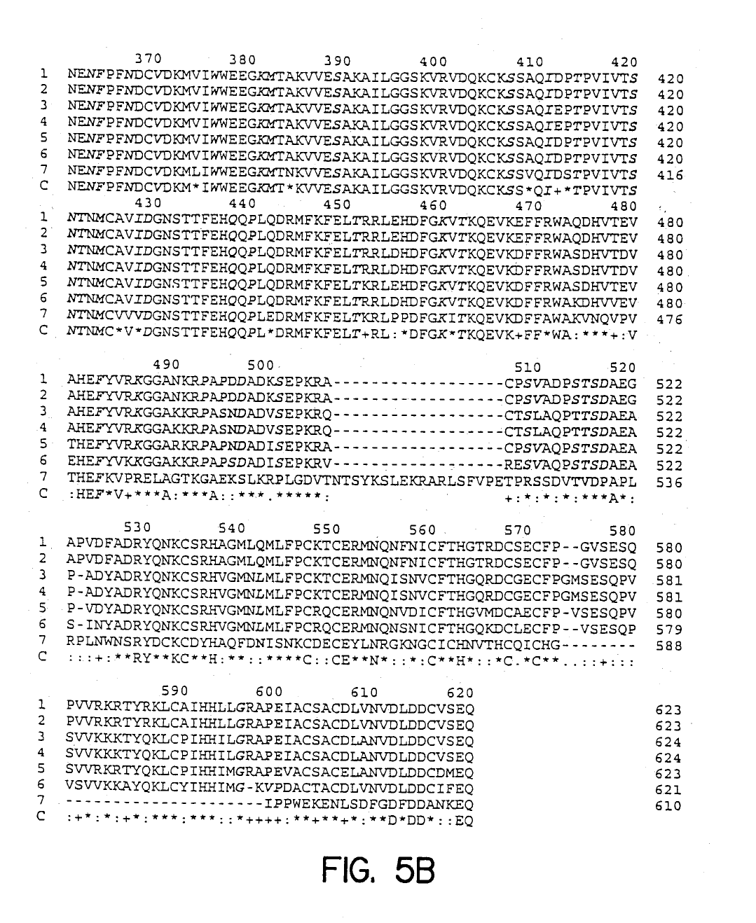

For the alignments presented herein (see, FIG. 5) for the AAV serotype, the CLUSTALW program was employed with parameters set as follows: scoring matrix BLOSUM, gap open 10, gap extend 0.1, gap distance 40% and transitions/transversions 0.5; specific residue penalties for hydrophobic amino acids (DEGKNPQRS), distance between gaps for which the penalties are augmented was 8, and gaps of extremities penalized less than internal gaps.

As used herein, a “corresponding” position on a protein, such as the AAV rep protein, refers to an amino acid position based upon alignment to maximize sequence identity. For AAV Rep proteins an alignment of the Rep 78 protein from AAV-2 and the corresponding protein from other AAV serotypes (AAV-1, AAV-6, AAV-3, AAV-3B, AAV-4, AAV-2 and AAV-5) is shown in FIG. 5. The “hit” positions are shown in italics.

As used herein, the term at least “90% identical to” refers to percent identities from 90 to 100% relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, it is also understood that the terms substantially identical or similar varies with the context as understood by those skilled in the relevant art.

As used herein, genetic therapy involves the transfer of heterologous nucleic acids to the certain cells, target cells, of a mammal, particularly a human, with a disorder or conditions for which such therapy is sought. The nucleic acid, such as DNA, is introduced into the selected target cells in a manner such that the heterologous nucleic acid, such as DNA, is expressed and a therapeutic product encoded thereby is produced. Alternatively, the heterologous nucleic acid, such as DNA, may in some manner mediate expression of DNA that encodes the therapeutic product, or it may encode a product, such as a peptide or RNA that in some manner mediates, directly or indirectly, expression of a therapeutic product. Genetic therapy may also be used to deliver nucleic acid encoding a gene product that replaces a defective gene or supplements a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product may be modified prior to introduction into the cells of the afflicted host in order to enhance or otherwise alter the product or expression thereof. Genetic therapy may also involve delivery of an inhibitor or repressor or other modulator of gene expression.

As used herein, heterologous or foreign nucleic acid, such as DNA and RNA, are used interchangeably and refer to DNA or RNA that does not occur naturally as part of the genome in which it is present or which is found in a location or locations in the genome that differ from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is expressed. Any DNA or RNA that one of skill in the art would recognize or consider as heterologous or foreign to the cell in which it is expressed is herein encompassed by heterologous DNA. Heterologous DNA and RNA may also encode RNA or proteins that mediate or alter expression of endogenous DNA by affecting transcription, translation, or other regulatable biochemical processes. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and DNA that encodes other types of proteins, such as antibodies.

Hence, herein heterologous DNA or foreign DNA, includes a DNA molecule not present in the exact orientation and position as the counterpart DNA molecule found in the genome. It may also refer to a DNA molecule from another organism or species (i.e., exogenous).

As used herein, a therapeutically effective product introduced by genetic therapy is a product that is encoded by heterologous nucleic acid, typically DNA, that, upon introduction of the nucleic acid into a host, a product is expressed that ameliorates or eliminates the symptoms, manifestations of an inherited or acquired disease or that cures the disease.

As used herein, isolated with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compounds can be substantially purified by the one-step method described in Smith and Johnson, Gene 67:31-40 (1988). The terms isolated and purified are sometimes used interchangeably.

Thus, by “isolated” is meant that the nucleic acid is free of the coding sequences of those genes that, in the naturally-occurring genome of the organism (if any) immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange change chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, receptor refers to a biologically active molecule that specifically binds to (or with) other molecules. The term “receptor protein” may be used to more specifically indicate the proteinaceous nature of a specific receptor.

As used herein, recombinant refers to any progeny formed as the result of genetic engineering.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated, such as tissue-specific regulation or induction by selected exogenous or endogenous factors.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. For gene expression a DNA sequence and a regulatory sequence(s) are connected in such a way to control or permit gene expression when the appropriate molecular, e.g., transcriptional activator proteins, are bound to the regulatory sequence(s).

As used herein, production by recombinant means by using recombinant DNA methods means the use of the well known methods of molecular biology for expressing proteins encoded by cloned DNA, including cloning expression of genes and methods, such as gene shuffling and phage display with screening for desired specificities.

As used herein, a splice variant refers to a variant produced by differential processing of a primary transcript of genomic DNA that results in more than one type of mRNA.

As used herein, a composition refers to any mixture of two or more products or compounds. It may be a solution, a suspension, liquid, powder, a paste, aqueous, non-aqueous or any combination thereof.

As used herein, a combination refers to any association between two or more items.

As used herein, substantially identical to a product means sufficiently similar so that the property of interest is sufficiently unchanged so that the substantially identical product can be used in place of the product.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Other forms of expression vectors, such as artificial chromosomes, that serve equivalent functions also are contemplated.

As used herein, vector is also used interchangeable with “virus vector” or “viral vector”. In this case, which will be clear from the context, the “vector” is not self-replicating. Viral vectors are engineered viruses that are operatively linked to exogenous genes to transfer (as vehicles or shuttles) the exogenous genes into cells.

As used herein, transduction refers to the process of gene transfer and expression into mammalian and other cells mediated by viruses. Transfection refers to the process when mediated by plasmids.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, referred to as a single nucleotide polymorphism (SNP), the identity of which differs in different alleles. A polymorphic region can also be several nucleotides in length.

As used herein, “polymorphic gene” refers to a gene having at least one polymorphic region.

As used herein, “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

As used herein, the term “gene” or “recombinant gene” refers to a nucleic acid molecule comprising an open reading frame and including at least one exon and (optionally) an intron sequence. A gene can be either RNA or DNA. Genes may include regions preceding and following the coding region (leader and trailer).

As used herein, “intron” refers to a DNA sequence present in a given gene which is spliced out during mRNA maturation.

As used herein, “nucleotide sequence complementary to the nucleotide sequence set forth in SEQ ID NO: x” refers to the nucleotide sequence of the complementary strand of a nucleic acid strand having SEQ ID NO: x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO: x refers to the complementary strand of the strand having SEQ ID NO: x or to any nucleic acid having the nucleotide sequence of the complementary strand of SEQ ID NO: x. When referring to a single stranded nucleic acid having the nucleotide sequence SEQ ID NO: x, the complement of this nucleic acid is a nucleic acid having a nucleotide sequence which is complementary to that of SEQ ID NO: x.

As used herein, the term “coding sequence” refers to that portion of a gene that encodes an amino acid sequence of a protein.

As used herein, the term “sense strand” refers to that strand of a double-stranded nucleic acid molecule that has the sequence of the mRNA that encodes the amino acid sequence encoded by the double-stranded nucleic acid molecule.

As used herein, the term “antisense strand” refers to that strand of a double-stranded nucleic acid molecule that is the complement of the sequence of the mRNA that encodes the amino acid sequence encoded by the double-stranded nucleic acid molecule.

As used herein, an array refers to a collection of elements, such as nucleic acid molecules, containing three or more members. An addressable array is one in which the members of the array are identifiable, typically by position on a solid phase support or by virtue of an identifiable or detectable label, such as by color, fluorescence, electronic signal (i.e. radiofrequency (RF), microwave or other frequency that does not substantially alter the interaction of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which may be any shape, including random shapes, needles, fibers, and elongated. Roughly spherical “beads”, particularly microspheres that can be used in the liquid phase, are also contemplated. The “beads” may include additional components, such as magnetic or paramagnetic particles (see, e.g., Dyna beads (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and may be order of cubic microns. Such particles are collectively called “beads.”

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. High Throughput Process

Provided herein are high throughput process for the generation of and identification of proteins that exhibit desired phenotypes and nucleic acid molecules, such as cis acting regulatory sequences, that have desired properties. The processes, include methods that are particularly adapted for high throughput protocols, which require accurate methods for identifying modified proteins.

A general directed evolution process includes the following steps:

1. Generation of diversity at the nucleic acid level, on the gene to be ‘evolved’

2. phenotypic characterization of the gene variants generated; and

3. identification of optimized gene variants.

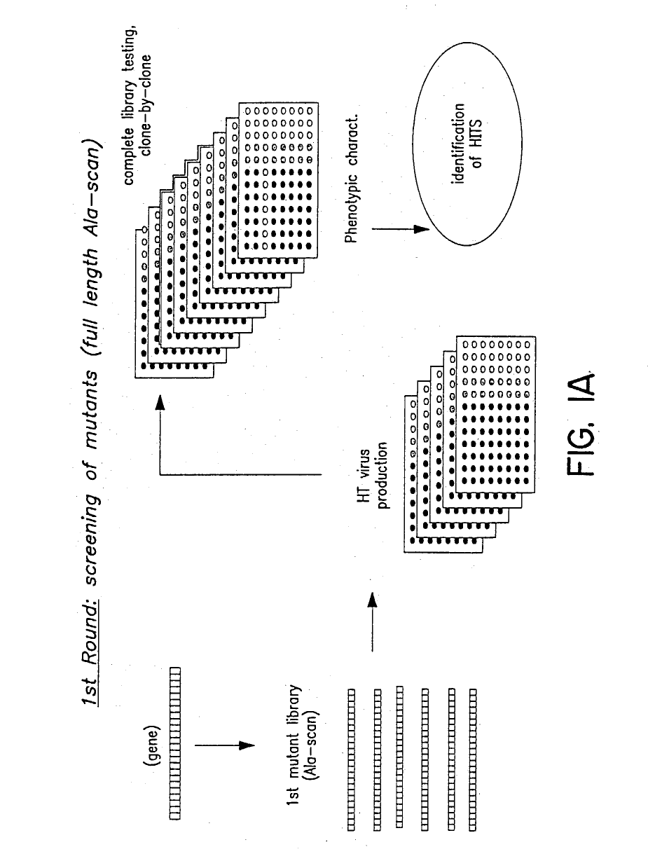

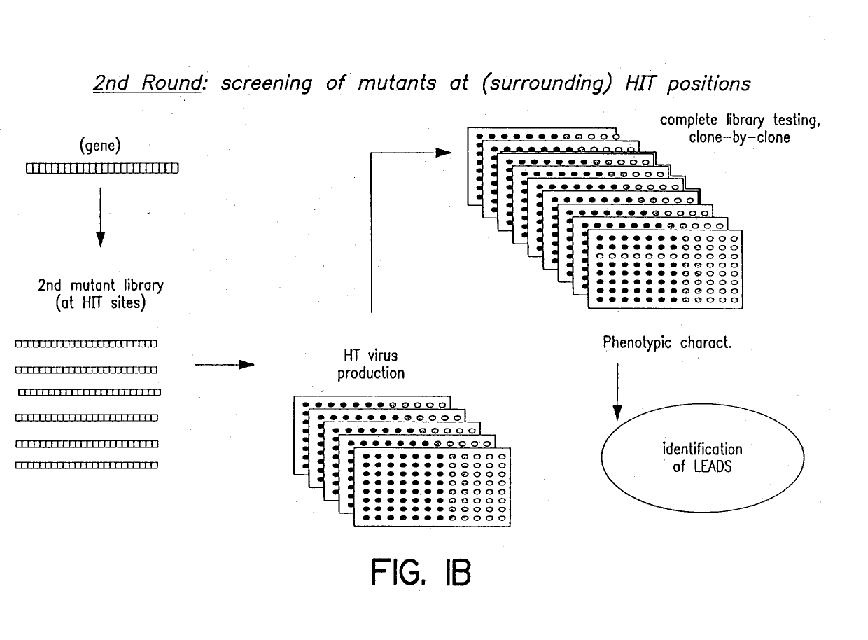

The processes provided herein effect these steps such that they are performed in a high throughput format (see, FIG. 1) that is optionally automated. A distinguishing characteristic of the processes provided herein, is that each candidate nucleic acid molecule is separately generated and screened. In an automated process at least some of the steps are performed without human intervention and are generally controlled by software. Most, if not all steps, are performed in addressable formats, such as at discrete locations in or on solid supports, such as microtiter plates or in other addressable formats, such as linked to coded supports. The supports can be electronically, physically, chemically or otherwise identifiable, such as by an identifiable symbology, including a bar code, or can be color coded. The methods are described generally with reference to proteins, which methods are the subject of co-pending U.S. application Ser. No. 10/022,249.

As described below (section D), the methods provided herein adapt such methods for use on nucleic acids by systematically altering the nucleotide sequences of nucleic acids, particularly DNA, such as cis acting sequences, and then by looking for alterations in function thereof. Such nucleic acids contemplated for such rational directed evolution include, but are not limited to, cis acting regulatory sequences, such as promoters, enhancers, silencers and other such sequences, and antisense and RNA.

1. Generation of Diversity Using a Semi-Rational Approach

A semi-rational approach to creating diversity or evolving genes is provided herein. The goal is to create diversity but to decrease the number of molecules to be screened. By reducing the numbers, the molecules can be screened in high throughput format molecule-by-molecule (or groups thereof).

Generation of diversity at the nucleic level, in principle, can be accomplished by a number of diverse technologies, such as, but not limited to, mutagenesis (either site-directed or random), recombination, shuffling and de-novo synthesis. These different technologies differ in the degree of diversity they generate as well as in the minimal length of the unitary change they can introduce (from single base to large domains). The outcome of step 1 is a collection of diverse, although highly related, molecules that constitutes what a ‘library’.

Generation of the library is important, since it provides the initial conditions for the process and is determinative of the outcome. The chances of finding an optimized gene version in a library is a function of the total diversity present in the library. In addition, the type of diversity introduced (such as, but not limited to, single point mutations, multiple point mutations, scarce small rearrangements, recombination of large domains, multiple shuffling) condition the outcome, particularly with respect to the generation of new variants compared to the original gene, and the probability that the new variants, not only exhibit the “evolved” function or property, but also work in their natural biological networks where they are expected to act by interacting, recognizing, and/or being recognized, by a large panoply of other proteins and other molecules.

Rapid Discovery of Protein Variants at the Amino Acid Level by Rational Mutagenesis (aa-scan)

A method, referred to herein as an amino-acid scan method for directed evolution, has been developed for generating protein variants (see, U.S. application Ser. No. 10/022,249, and corresponding International PCT application No. PCT/IB02/03921). This method can be performed on an entire protein or selected domains thereof, or can be used to identify benchmark sequences, such as functional domains, and, for example, recombine them as exchangeable units or restrict the diversity to limited or specific regions of the protein. Not only can this method be used with the processes provided herein, but it also has applications for any methods that use such variants or require generation of such variants, such as, but not limited to, searches for consensus sequences and homology regions that are used in functional genomics, functional proteomics; comparative modeling in protein crystallography and protein modeling; searches for natural diversity, (e.g., directed evolution methods in U.S. Pat. Nos. 6,171,820, 6,238,884, 6,174,673, 6,057,103, 6,001,574, 5,763,239); exon- or family-shuffling-based diversity (e.g., directed evolution using gene shuffling (see, e.g., U.S. Pat. Nos. 6,096,548, 6,117,679, 6,165,793, 6,180,406, 6,132,970); the optimization of only the CDRs regions (e.g., directed evolution of antibodies see., e.g., U.S. Pat. Nos. 5,723,323, 6,258,530, 5,770,434, 5,862,514) and other methods (see, e.g., U.S. Pat. Nos. 5,837,500, 5,571,698, 6,156,509).

The amino-acid scanning-based method has advantages that prior methods do not have. For example, prior methods are based upon the underlying assumption that there are parts of the molecule (gene or protein) that are sufficiently adapted to perform their respective function, and further changes are not advantageous. Such methods do not look at total potential plasticity of a given molecule, but at the plasticity still permitted while keeping some basic functions in place. By choosing this route, however, additional potential variation is missed. The potential in the intrinsic plasticity of those regions that are presumed ‘preserved’ is lost. For instance, methods (e.g., those in U.S. Pat. Nos. 6,171,820, 6,238,884, 6,174,673, 6,057,103, 6,001,574, 5,763,239) that use natural diversity can miss the potential plasticity within those regions that are naturally ‘conserved’, i.e. where there is no natural diversity. Methods that rely on exon- or family-shuffling-based diversity can miss the potential plasticity within regions contained in the shuffled fragments, i.e. within the fragments exchanged as a block. The method provided herein in contrast is sufficiently flexible to create mutants at a variety of levels, including at the single amino acid level; i.e. the method can generate mutants that differ from each other at a single amino and not at a larger block level. The challenge solved by the method herein is to generate diversity at the single amino acid level, without moving too close to a pure ‘random’ approach, which results in an intractable number of mutants.

The method provided herein is based on the premise that there are single amino acids or small blocks of sequence of amino acids that are either (1) directly involved in the activities that the methods ‘evolve’ (these amino acids would be at or close to the ‘active sites’ of the protein); or (2) directly involved in maintaining within the protein the intra-molecular environment that allows the active site(s) to stay active.

Potential plasticity at the amino acid level can be exploited if amino acids or blocks of amino acids directly involved in the active sites for the activity to be evolved are known. Often they are not known. The problem that is solved herein, however, is how to exploit the potential plasticity at the amino acid level when nothing is known about the structure of the protein in question or about the position of its single or numerous active sites.

The technology referred to herein as amino acid-scanning has been used to precisely identify those amino acids directly involved in the active sites of some enzymes and receptors (see, e.g., Becl-Sickinger et al. (1994) Eur. J. Biochem. 223:947-958; Gibbs et al. (1991) J. Biol. Chem. 266:8923-8931; Matsushita et al. (2000) J. Biol. Chem. 275:11044-11049) but has not been employed for directed evolution or for the generation of diversity. The amino acid scan as practiced in the prior art is used to produce a set of protein mutants, often within the region suspected to contain the active site(s), such that in each individual mutant a selected residue, such as Ala, replaces a different amino acid. Ala or other neutral amino acids generally, although not necessarily, is selected as a replacement amino acid since, except in instances in which the replaced amino acid is directly involved in an active site, it should have a neutral effect on the protein activity and not disturb the native secondary structure of the protein. In instances in which the replaced amino acid is directly involved in an active site the activity of that site is lost or altered. Amino-acid scanning, such as Ala-scanning, has been successfully applied for the identification of active sites in a number of proteins, and has been performed in computer-based rational drug design methods. Other amino acids, particularly amino acids that have a neutral effect, such as Glycine, can also be used.

The amino acid scanning method is employed herein for the generation of the mutant proteins for screening for identification of sites or loci in a target protein or regions in a protein that alter a selected activity. In performing this method, the amino acids are each replaced, one-by-one along the full-length of the protein, or one-by-one in pre-selected domains, such as domains that possess a desired activity or exhibit a particular function. Once sites of interest are identified other methods for generating diversity from the resulting molecules can be employed or the further steps of the method provided herein can be performed.

The method includes the following steps:

(1) Identification of the active site(s) on the full length protein sequence. In one embodiment a full-length amino acid-scan, typically, although not necessarily, an Ala-scan, or the identification and positioning of the active site(s) on proteins of either known or unknown function. For purposes herein, an active site is not necessarily the natural active sites involved in the natural activity of a target protein, but those amino acids involved in the activities of the proteins under ‘directed evolution’ with the purpose of either gain, improvement or loss of function.

The whole process of the ‘identification of the active site(s) on the full length protein sequence requires the following sub-steps:

a. Generation of a mutant library (on the gene to be evolved) in which each individual mutant contains a single mutation located at a different amino acid position and that includes a systematic replacement of the native amino acid by Ala or any other amino acid (always the same throughout the entire protein sequence);

b. phenotypic characterization of the individual mutants, one-by-one and assessment of mutant protein activity;

c. identification of those mutants that display an alteration, typically a decrease, in the selected protein activity, thus, indicating that amino acids directly involved in the active site(s) have been hit. The aa positions whose aa-scan mutations display an alteration, typically a loss or decrease, in activity are named HITS.

The identification of the active site(s) (HITS) is thus, by this method, made in a completely unbiased manner. There are no assumptions about the specific structure of the protein in question nor any knowledge or assumptions about the active site(s). The results of the amino acid scan identify such sites.

Once the active site(s) (the HITS) has (have) been identified, those amino acids either at or surrounding the active sites, such as within 1, 2, 3, . . . 10, 20 or any selected regions, as the unitary elements of exchange and generate diversity either at or around one of those sites or as a combined diversity at several sites at a time can be assessed. This process includes the following steps:

a. Generation of a new mutant library (on the gene to be evolved) in which each individual mutant contains either single or multiple mutations located at (or surrounding) a specific active site (a HIT) position detected by the precedent α-scan process. In the example these mutations include replacement, in each individual mutant, of the native amino acid located either at (or surrounding) the HIT position by one of all other possible amino acids, such that, in the library, and at (or surrounding) each HIT position the native amino acid has been replaced by all possible amino acids.

b. Identification of those mutants that display an increase in protein activity, thus indicating that a new sequence at or surrounding an active site has been identified with higher activity compared to the native sequence. These optimized sequences are named LEADS.

The process can be repeated as many times as desired, in search for new combinations of optimal amino acids at (or surrounding) the different HIT sites. Each time, the process includes the steps of generating of a new mutant library (of the gene to be evolved) in which each individual mutant contains either single or multiple mutations located at (or surrounding) a specific active site (a HIT) position; phenotypic characterization of the individual mutants, one-by-one and assessment of mutant protein activity; and identification of those mutants that display an increase in protein activity, thus indicating that a new sequence at or surrounding an active site has been identified with higher activity compared to the native sequence. These optimized sequences are again named LEADS (second generation LEADS).

2. Phenotypic Characterization of the Gene Variants

This step requires the expression of the gene variants in order to allow them to manifest their respective phenotypes. Gene expression can be accomplished by different means: in vitro, in reconstituted systems or in vivo in cellular systems, including bacterial and eukaryotic cells. For exemplification purposes, reference is made to in vivo systems. Those of skill in the art can readily adapt these methods for in vitro systems, including those using biochemical assays.

This step is important step for several reasons:

(a) Expression System and Protein Processing.

Depending on the system used (either bacteria or eukaryotic cells), as well as on the specific gene to be ‘evolved’, the variant proteins may or may not be appropriately processed, especially when post-translational modifications are involved, and therefore be able or not to elicit their potential activity. Consequently, the expression system (bacteria vs. eukaryotic cells) has to be carefully chosen.

(b) Standardization of the Expression System.

The technologies available for gene transfer and expression into either bacteria or eukaryotic (let's consider mammalian) cells widely vary in their intrinsic efficiencies. While it is very easy to efficiently transfer and express genes in bacteria by chemical/physical methods (transformation), that is not the case for mammalian cells, where the transformation (here called transfection) process is inefficient and unreliable, specially when reproducibility and robustness are necessary in miniature, large number—and small scale high throughput settings like those necessary to analyze gene variant libraries. Therefore, when transfection is used on mammalian cells, the specific activity measured for the individual variants in the library most probably does not accurately reflect the real specific activity of the molecules involved. As provided herein, transduction, the process of gene transfer and expression into mammalian and other cells mediated by viruses, overcomes the limitations of transfection.

(3) Characterization

A distinction must be made between the ‘expression’ of the gene variants and their ‘phenotypic characterization’. The expression system (either bacteria or mammalian cells) is only a vehicle to convert the gene variants into protein variants. The phenotypic characterization is performed on the protein variants, and may have nothing to do, depending on the specific system under study, with the cellular system used to express the variants. The phenotypic characterization requires the use of specific assays (either biochemical (cell-free) or cell-based assays) in which the activity of the different cell mutants is challenged and assessed. In addition to the implications discussed below, these assays must be designed in such a way that they reflect the final environment in which the ‘evolved’ protein is expected to act. As an example, when optimizing an enzyme to be used in an artificial industrial setting, the assay should reproduce those conditions (temperature, pH, media composition . . . ) of the real-life industrial reaction mixture, which may be relatively easy to do. When the final destination of the ‘evolved’ protein is a complex biological setting, such as the intracellular environment, the extra-cellular milieu (example: circulating proteins) or the structure of a virus, the necessary assay(s) may be quite difficult to setup. With a few exceptions, most of the work done so far on directed evolution has been made on simple enzymes for which all the necessary settings are relatively easy to implement.

Methods for Accurately Titering Viruses

Much progress in gene therapy, genomics, biotechnology and, in general, biomedical sciences, depends on the ability to generate and analyze large numbers and small amounts of specific viruses. High throughput technologies are employed in disciplines such as functional genomics and gene therapy in which the use of viruses plays a key role for the efficient transfer and analysis of large collections of genes or libraries. Also, virus samples and biomedical samples containing viruses are routinely analyzed in thousands of hospitals, health centers, academic labs and biotech setting.

Furthermore in processes herein, accurate titration can be important in at least two steps in the process. After preparation of the viruses with the mutated variant, and prior to screening, it is necessary to know the concentration of titer of the viruses in the sample, so that results among the samples can be compared. The methods in this section designated Real Time Virus Titering (RTVT™; and Tagged Replication and Expression Enhancement Technology (TREE™) are advantageously used (for discussions of RTVT™ and TREE™ see, International PCT application No. PCT/FR01/01366 published as International PCT application No. WO 01/186291, EP 1281081, FR Patent 2802645, EP 1244912 and International PCT application No. WO 01/44809 and the EXAMPLES below).

The methods in this section are also used in data analysis when measuring the output signal. As described below, output signal can be assessed by a Hill analysis or a second order polynomial or other algorithm that describes the interaction of biological molecules in complex system. In addition, where the output signal is actually the number of viral particles or ip produced, the methods in this section RTVT and TREE are advantageously used.

Prior art virus titration methods (RCA, dRA . . . ),for determining the amount of virus present in a biological sample, are based on the assessment of some kind of output signal, such as cytopathic effect, lysis or plaques and cell fusion focuses, induced in a reporter cell following a fixed time after infection with varying concentrations of the sample containing the virus. The lowest concentration of the sample at which no signal can be measured is taken as the titer of the virus in that sample. These approaches are known as “serial dilution” or “limiting dilution” methods. In limiting dilution methods, one single virus concentration, measured at a given time end-point gives rise to a single measurement of the output signal. These methods tend to be destructive in that assessment destroys the reaction so that only a single measurement can be taken on a sample.

Real Time Virus Titering (RTVT™)

When a virus infects a cell, the infected cell undergoes a number of changes that can be followed over time and quantified. Such changes are designated herein as the “output signal”. The cell reports an output signal in response to the infection and, therefore, it is named here a reporter cell. One such output signal, is, for example, the expression of the genes carried by the virus (whether they are viral genes or exogenous genes (transgenes)). The output signal (for instance the level of expression of those genes) develops over time and depends, mainly, on two factors: i) the time point (“t”) at which its level is measured after infection and ii) the amount of virus infecting the cell; i.e. the concentration of the virus preparation used to infect the cell (“s”). The output signal, at a given time point after infection, is higher for higher concentrations of the virus infecting the reporter cells; and for any given concentration of virus, the output signal increases with time after infection until it generally reaches a plateau level.

Real Time Virus Titering (RTVT) published as International PCT application No. WO 01/186291, which is based on PCT/FR01/01366 and EXAMPLES below) uses non-destructive methods for the assessment of output signal. Real Time Virus Titering is a viral titration method based on the kinetic analysis of the development of the output signal in virus-infected cells, tested at a single concentration of virus or biological sample. Instead of fixing the time point after infection and varying the concentration of the sample as is done in limiting dilution methods, in the Real Time Virus Titerng RTVT™ method, a fixed concentration of virus is used and the generation of a signal over time is assessed. Hence the signal is measured as a function of time at a single virus concentration. In this situation, a single virus sample (concentration), whose output signal is measured at a number of time points, can give rise to as many measurements of the output signal as needed, and, eventually to a continuous, over time, assessment of the signal in real time.

Real Time Virus Titerng RTVT™ can be advantageously used in high throughput methods in which large numbers of biological samples are analyzed at the same time. This is the case, for instance, when titering viruses in a virus library. Limiting dilution methods rely on the output signal generated by a number of dilutions of each individual sample. If, for example, 10 dilutions (or experimental points) of each virus are used for a titration using a limiting dilution method, the analysis of a library containing 10,000 viruses require analysis of 10⁵ (i e., 10×10,000) experimental points. The Real Time Virus Titerng RTVT™ method requires only one dilution per sample, thereby requiring 10-fold fewer experimental points than a limiting dilution method. For a Real Time Virus Titerng RTVT™ titering system, the time (tβ) necessary for the output signal to reach a reference value (β) is a direct function of the concentration of virus. Thus, tβ can be used to directly determine the concentration of the virus.

A limitation of the Real Time Virus Titerng RTVT™ limiting dilution titering method, however, is that not all the viruses (nor the genes carried by the viruses) generate a readily measured output signal that can be followed over time using non-destructive methods.

Tagged Replication and Expression Enhancement (TREE)

A method for titering designated Tagged Replication and Expression Enhancement Technology (TREE™) is provided herein. This system includes: i) a cell, ii) a reporter virus carrying a reporter gene, whose activity can be followed over time by a non-destructive method (i.e., fluorescence), iii) the virus (or virus library to be titered), herein referred to as the “titering virus”. The elements are employed such that the virus to be titered interferes with any output signal generated by the reporter virus, leading to either decrease or increase in the amount of that signal. The higher the amount of virus to be titered, the higher is the interference with the reporter virus and output signal. In the absence of virus to be titered, the kinetics of the output signal generated by the reporter virus are followed using the Real Time Virus Titerng RTVT™ titering method. In the presence of increasing amounts of the virus to be titered the output signal takes longer (or shorter) to develop as a function of the amount of virus to be titered.

Using the TREE™ titering method, tβ, the time necessary for the output signal to reach a reference value (β) is a direct function of the concentration of virus and, therefore, tβ can be used to determine the concentration of the virus to be titered. It is demonstrated herein (see the EXAMPLES) that when using the TREE system for titering, once an appropriate reference value (β) is determined for the output signal generated from the reporter virus, the time tβ is a function of the concentration of the virus being titered (see Example). Therefore, the concentration (titer) of the virus to be titered, can be assessed by assessing the change induced in tβ by an aliquot of the virus to be titered. In a calibrated TREE titering assay, only one aliquot virus to be titered is needed to determine its titer, which is determined by measuring the shift in the tβ of the system. The only condition is that the virus to be titered must “interfere” (i.e., increase or decrease) the output signal of the reporter virus.

A calibration curve representing tβ vs. the amount of virus to be titered is obtained using aliquots of a reference batch of virus of known titer (previously determined using any titering procedure). The calibration curve can then be used to determine the amount of virus in a sample of unknown titer, based on the change caused by an aliquot of the sample on the tβ of the system and the corresponding titer read from the calibration curve.

3. Identification of Gene Variants

There are at least two considerations in this step:

(a) Selection vs. Screening.

Depending on the specific protein involved, and under certain and very specific assay conditions, those variants that have been ‘evolved’ may elicit a selective advantage over the native version. This situation represents the most simple case: the cells (bacteria or mammalian) expressing the library of protein variants, as a pool or mixture, can be exposed to the selective conditions, which evidence the best optimized variants. This situation is however very rare and difficult to achieve. It's difficult if not impossible to set up a suitable ‘selective’ assay for any protein that one may want to optimize. For the vast majority of the cases, selection will not be possible. Therefore pools of molecules cannot be used, because the specific readouts of the assays could not be attributed to individual variants. When the simplistic selection approach is not possible, then two things are absolutely needed: (a) a ‘one-by-one’ approach, i.e. each individual variant must be physically separated from the others and its activity tested independently; (b) an accurate and quantitative analysis that can distinguish slight differences in activity among the different variants along a wide range of performance values.

(b) Accurate Quantitative Analytics

When selection is not possible, the optimized variants must be distinguished from the native variant otherwise. The different degrees of optimization among the different variants in the library should, in addition, be distinguished if those variants showing the highest optimization level are to be identified. A powerful quantitative analytical protocol is then mandatory. These analytics should be able to attribute quantitative features (on the activity tested in the specific assay) to each of the variants tested and to rank them according to their individual performance. This requires, in addition, that each variant in the library is assayed individually; the use of pools or mixtures of molecules would hamper the ability to identify the right variants.

For such analysis, the output signal can be assessed by a Hill analysis (see Examples and (published International PCT application No. WO 01/44809 based on PCT No. PCT/FR00/03503),) or a second order polynomial (see, Examples and (Drittanti et al. (2000) Gene Ther. 7: 924-929)) or other algorithm that describes the interaction of biological molecules in complex system, such as the interaction between cells and biological agents. In addition, where the output signal is actually the number of viral particles or ip produced, the methods in this designated Real Time Virus Titering (RTVT™) and Tagged Replication and expression enhancement (TREE™) are advantageously used (for a discussion of RTVT™, see, International PCT application No. PCT/FR01/01366 published as International PCT application No. WO 01/186291 and the EXAMPLES below) or a refinement of that method provide herein and designated Tagged Replication and expression enhancement (TREE™) described above and in the examples.

C. Practice of the Process

In one embodiment, the process provided herein includes the following steps.

1. Generation of Diversity or Source of Existing Diversity

Generation of a plasmid library containing the genetic variants. The genetic variants are physically separated from each other. Any model such as, but not limited to, amino acid scanning, mutagenesis, or recombination may be used to generate the plasmid library.

2. Expression of the Genetic Variants

Any method for expression of variants is contemplated. In particular the following alternatives are particularly suitable for high throughput performance.

a. Expression in Bacterial Hosts

The mutated forms of the nucleic acid are prepared or introduced into plasmids for expression in bacterial cells. The genetic variants are expressed from suitable bacterial cells, which are prepared by transformation aliquots of the cells with each member of the plasmid library (each genetic variant continues to be physically separated from each other).

b. Expression in Eukaryotic Host Cells

A virus library is generated from the plasmid library. The virus library, in which each different member is separately maintained, is prepared by:

(1) Transfection of the plasmid library into appropriate virus-producer cells (viruses produced, each one carrying a different genetic variant present in the original plasmid library, are physically separated from each other);

(2) Titration of the virus library (of each individual virus present in the library, separately). Titration is effected by any method, but generally by either a method designated Real Time Virus Titering (RTVT™) (see, International PCT application No. PCT/FR01/01366 published as International PCT application No. WO 01/186291 and the EXAMPLES below) or a refinement of that method provide herein and designated Tagged Replication and expression enhancement (TREE™) described above and in the examples;

(3) Standardization of the virus library to equal concentrations of all the individual viruses in the library (individual viruses continue to be physically separated from each other);

(4) Expression of the genetic variants from appropriate mammalian cells by transduction with the virus library (each genetic variant continues to be physically separated from each other and each individual virus is handled separately from the others).

3. Phenotypic Characterization of the Variant Proteins.

The variant proteins are expressed (from either plasmids in bacterial cells (step 2) or viruses in mammalian cells (step 4)) and their activity is assessed in one or more appropriate specific assays. The assays can be both types: biochemical (cell-free) assays and/or cell-based assays. The variant proteins in the library are physically separated from each other and their activity is individually assessed on a one-by-one basis.

The assays can be performed in one of a variety of ways, including, but are not limited to:

a. Using a single-point dilution for each individual variant protein, followed by a kinetic analysis (multiple time points) of the read-out by technologies like Tagged Replication and expression enhancement (TREE™), or any other appropriate technology

b. Using serial dilutions of each individual variant protein, followed by, for example, the Hill-based analysis of the read-out by technologies or any other appropriate technology. Hill based analyses assess the interaction between cells and biological agents (see, published International PCT application No. WO 01/44809 based on PCT No. PCT/FR00/03503).

The goal of these methods is to identify proteins having an evolved function or property.

Lead Identification

Based on the results obtained from the assays described above, each individual protein variant is individually tested for the parameters that assess the activity, property, function or structure of interest. Variants are ranked out according to their activity features. Those variant proteins best suited for the specificities of each individual project and system under study are then selected. The selected leads can be used for the desired purpose or further evolved or mutated to achieve desired activities.

Typically, as for most directed evolution methods, the process is an iterative one, in which mutated variants are produced, screened, the best identified and then selected. The selected variants are then subjected to further evolution and the screening process repeated. This is repeated until the desired goal is achieved.

This further evolution may employ the methods herein or any directed evolution method or combinations thereof. The methods for variant production include the amino-acid scan method herein, which provides a rational approach to variant generation. Other rounds can include combinations of any other method for directed evolution known and/or combinations thereof.

D. Directed Evolution of Nucleic Acid Molecules

The methods exemplified for use with proteins are adapted herein for use with nucleic acid molecules. In the protein methods, each position in protein is first altered by substituting each amino acid for one other, such as Ala. This is done by preparing nucleic acid molecules that encoded the mutant proteins and then the nucleic acid molecules are each expressed.

For identification of nucleic acid molecules exhibiting altered expression, the process is substantially the same, except that each base is systematically altered and the function of the resulting molecule is assayed. For example, if a promoter is altered, it can be linked to protein-encoding nucleic acid and expression of the encoded protein assessed. For altering functional nucleic acid molecules, each base is systematically changed to a different base, such as each changed to A, or each changed to A, and the A's, optionally, altered to something else, and hits are identified by monitoring the function of the resulting nucleic acid molecule. Hits are identified, and the bases at each hit position are changed to each of the other bases, and the resulting nucleic acid molecules are individually screened. Those that have desired properties, are selected as leads. The leads can be combined to produce “super” leads that have two or more of the identified changes of interest until a nucleic acid molecule that has the desired properties is identified.

Generally functional nucleic acid molecules include cis acting regulatory regions, such as promoters, enhancers, silencers and protein binding regions, such as transcriptional regulators. For example, a promoter of interest can be modified to produce a promoter that is regulatable by a selected condition or endogenous or exogenous factor, or, by selecting appropriate assays, it can be rendered tissue specific, or constitutive or “stronger” in that it directs higher expression of the operatively linked encoded protein or preferential of the promoter compared to others in the same system.

For example, a region containing the target region of the nucleic acid molecule, such as a 100, 250, 500, 1000, 2000, 3000, 4000, 5000, 10,000 base pair region that includes the putative or identified regulatory region is identified and used for modification as described herein. The entire region or a portion thereof is modified and the function assayed.

Promoter Optimization

Three exemplary approaches (A, B, C) can be used for promoter optimization as described below. The mutagenesis can be done, for example, using one-by-one changses, point mutations and/or non combinatorial or non random (non stochastic) changes. Target promoters include any known to those of skill in the art, including, but not limited to, viral promoters, such as adenovirus, AAV, cytomegalovirus, herpes, vaccinia and retrovirus promoters, bacterial and bacteriophage promoters, cellular eukaryotic, including mammalian, promoters. Exemplary promoters include, but are not limited to, E1 from adenovirus, the adenovirus late promoter, the SV40 promoter; bacteriophage promoters, such as SP6, T4 and T7, cytomegalovirus promoters, such as the CMV early promoter, bacterial promoters, such as lacZ, antibiotic resistance, such as tetracycline resistance, alkaline phosphatase, and mammalian promoters such as phosphoglucokinase (PGK) and EF1 alpha (elongation-1 alpha). For screening, the promoter can be operatively linked to its native protein or to an endogenous protein of interest.

Approach A: ‘rational mutagenesis’.

The rational mutagenesis approach generally is a 3-step process. In the first step (base-scanning), single point mutations are introduced throughout the promoter region (regardless of presumed involvement in promoter activity) such that AT pairs are change into GC pairs and vice versa, one at a time. This process identifies base positions (called HITs) that are directly or indirectly involved in promoter activity. All four bases are tested at each individual HIT position. Each mutant contains a single base change. Mutants leading to higher expression are called LEADS.

In the next step, mutations found in different LEADS are combined on the same promoter molecule to generate what are designated SUPERLEADS, which are mutants with a variable number (more than one) of LEAD mutations.

Approach B: ‘consensus conversion’.

The initial consensus sequences are gradually converted by mutagenesis into corresponding consensus sequences known to have higher activity. The gradual conversion of the consensus sequences from ‘initial’ into optimal is made by the accumulation of single point mutations from the borders towards the middle of the consensus sequences. A collection of mutants is generated such that each mutant differs from the preceding mutant in, for example, an addressable collection, such as a positionally-addressable array, in a new mutation that has been added.

Approach C: “moving consensus”.

A collection of single base deletion mutants is generated such that each mutant either increases or decreases the distance between one or more consensus sequence in the promoter by virtue of insertions or deletions.

Any of these methods or other such methods can be used and then combined with the screening methods described in the preceding sections to identify nucleic acid molecules that have a desired activity. Similar methods can be employed to modify other regulatory regions of nucleic acid molecules as well coding regions. These methods also can be used to assess the function of regions of a targeted nucleic acid or to identify regulatory regions.

E. Directed Evolution of a Viral Gene or Regulatory Sequence

A process of directed evolution of a viral geneis exemplified in copending U.S. application Ser. Nos. 10/022,390 and 10/022,249. The methods as provided can be used to optimize, for example, a promoter for use in AAV for expression of a heterologous gene. In such instances, the nucleic acid and promoter is inserted into an AAV vector and expression of the protein monitored. Alternatively, the method can be used to optimize expression of an AAV promoter, such as a capsid or rep promoter for expression of an operatively linked heterologous gene.

Recombinant viruses have been developed for use as gene therapy vectors. Gene therapy applications are hampered by the need for development of vectors with traits optimized for this application. The high throughput methods provided herein are ideally suited for development of such vectors. In addition to use for development of recombinant viral vectors for gene therapy, these methods can also be used to study and modify the viral vector backbone architecture, trans-complementing helper functions, where appropriate, regulatable and tissue specific promoters and transgene and genomic sequence analyses. Recombinant AAV (rAAV) is a gene therapy vector that can serve as a model for application of the methods herein for these and other purposes.

Adeno-associated virus (AAV) is a defective and non-pathogenic parvovirus that requires co-infection with either adenovirus or herpes virus, which provide helper functions, for its growth and multiplication. There is an extensive body of knowledge regarding AAV biology and genetics (see, e.g., Weitzman et al. (1996) J. Virol. 70: 2240-2248 (1996); Walker et al. (1997) J. Virol. 71:2722-2730; Urabe et al. (1999) J. Virol. 23:2682-2693; Davis et al. (2000) J. Virol. 23:74:2936-2942; Yoon et al. (2001) J. Virol. 75:3230-3239; Deng et al. (1992) Anal Biochem 200:81-85; Drittanti et al. (2000) Gene Therapy 7:924-929; Srivastava et al. (1983) J. Virol. 45:555-564; Hermonat et al. (1984) J. Virol. 51:329-339; Chejanovsky et al. (1989) Virology 173:120-128; Chejanovsky et al. (1990) J. Virol. 64:1764-1770; Owens et al. (1991) Virology 184:14-22; Owens et al. (1992) J. Virol. 66:1236-1240; Qicheng Yang et al. (1992) J. Virol. 66:6058-6069; Qicheng Yang et al. (1993) J. Virol. 67:4442-4447; Owens et al. (1993) J. Virol. 62:997-1005; Sirkka et al. (1994) J. Virol. 68:2947-2957; Ramesh et al. (1995) Biochem. Biophy. Res. Com. Vol 210 (3), 717-725; Sirkka (1995) J. Virol. 69:6787-6796; Sirkka et al. (1996) Biochem. Biophy. Res. Com. 220:294-299; Ryan et al. (1996) J. Virol. 70:1542-1553; Weitzman et al. (1996) J. Virol. 70:2440-2448; Walker et al. (1997) J. Virol. 71:2722-2730; Walker et al. (1997) J. Virol. 71:6996-7004; Davis et al. (1999) J. Virol. 73:2084-2093; Urabe et al. (1999) J. Virol. 73:2682-2693; Gavin et al. (1999) J. Virol. 73:9433-9445; Davis et al. (2000) J. Virol. 74:2936-2942; Pei Wu et al. (2000) J. Virol. 74:8635-8647; Alessandro Marcello et al. (2000) J. Virol. 74:9090-9098). AAV are members of the family Parvoviridae and are assigned to the genus Dependovirus. Members of this genus are small, non-enveloped, icosahedral with linear and single-stranded DNA genomes, and have been isolated from many species ranging from insects to humans.

AAV can either remain latent after integration into host chromatin or replicate following infection. Without co-infection, AAV can enter host cells and preferentially integrate at a specific site on the q arm of chromosome 19 in the human genome.

The AAV genome contains 4975 nucleotides and the coding sequence is flanked by two inverted terminal repeats (ITRs) on either side that are the only sequences in cis required for viral assembly and replication. The ITRs contain palindromic sequences, which form a hairpin secondary structure, containing the viral origins of replication. The ITRs are organized in three segments: the Rep binding site (RBS), the terminal resolution site (TRS), and a spacer region separating the RBS from the TRS.

Regulation of AAV genes is complex and involves positive and negative regulation of viral transcription. For example, the regulatory proteins Rep 78 and Rep 68 interact with viral promoters to establish a feedback loop (Beaton et al. (1989) J. Virol 63:4450-4454; Hermonat (1994) Cancer Lett 81:129-136). Expression from the p5 and p19 promoters is negatively regulated in trans by these proteins. Rep 78 and 68, which are required for this regulation, have bind to inverted terminal repeats (ITRs; Ashktorab et al. (1989) J. Virol. 63:3034-3039) in a site- and stand-specific manner, in vivo and in vitro. This binding to ITRs induces a cleavage at the TRS and permits the replication of the hairpin structure, thus, illustrating the Rep helicase and endonuclease activities (Im et al. (1990) Cell 61:447-457; and Walker et al. (1997) J. Virol. 71:6996-7004), and the role of these non-structural proteins in the initial steps of DNA replication (Hermonat et al. (1984) J. Virol. 52:329-339). Rep 52 and 40, the two minor forms of the Rep proteins, do not bind to ITRs and are dispensable for viral DNA replication and site-specific integration (Im et al. (1992) J. Virol. 66:1119-112834; Ni et al. (1994) J. Virol. 68:1128-1138.

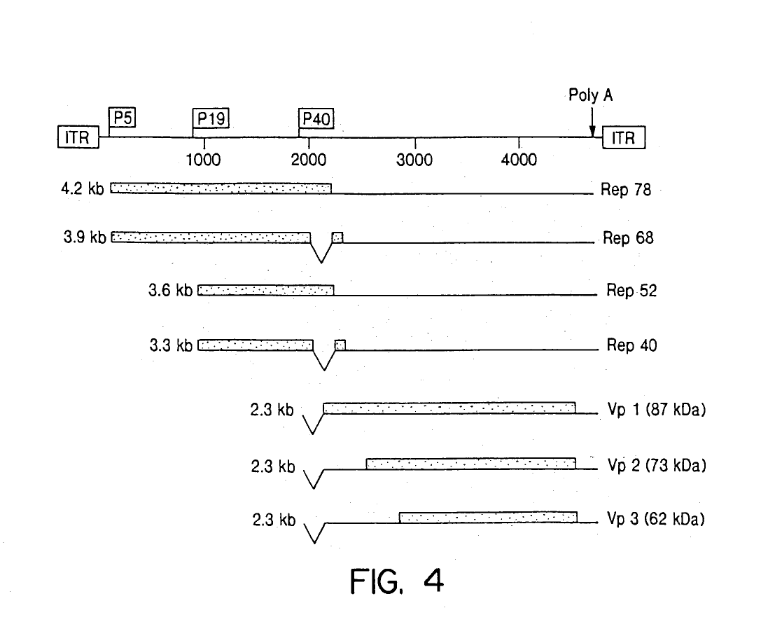

The genome (see, FIG. 4) is organized into two open reading frames (ORFs, designated left and right) that encode structural capsid proteins (Cap) and non-structural proteins (Rep). There are three promoters: p5 (from nucleotides 255 to 261: TATTTAA), p19 (from nucleotide 843 to 849: TATTTAA) and p40 (from nucleotides 1822 to 1827: ATATAA). The right-side ORF (see FIG. 4) encodes three capsid structural proteins (Vp 1-3). These three proteins, which are encoded by overlapping DNA, result from differential splicing and the use of an unusual initiator codon (Cassinoti et al. (1988) Virology 167:176-184). Expression of the capsid genes is regulated by the p40 promoter. Capsid proteins VP1, VP2 and VP3 initiate from the p40 promoter. VP1 uses an alternate splice acceptor at nucleotide 2201; whereas VP2 and VP3 are derived from the same transcription unit, but VP2 use an ACG triplet as an initiation codon upstream from the start of VP3. On the left side of the genome, two promoters p5 and p19 direct expression of four regulatory proteins. The left flanking sequence also uses a differential splicing mechanism (Mendelson et al. (1986) J. Virol 60:823-832) to encode the Rep proteins, designated Rep 78, 68, 52 and 40 on the basis molecular weight. Rep 78 and 68 are translated from a transcript produced from the p5 promoter and are produced from the unspliced and spliced form, respectively, of the transcript. Rep 52 and 40 are the translation products of unspliced and spliced transcripts from the p19 promoter.

The rep protein is a adeno-associated virus protein involved in a number of biological processes necessary to AAV replication. The production of the rRep proteins enables viral DNA to replicate, encapsulate and integrate (McCarty et al. (1992) J. Virol 66:4050-4057; Horer et al. (1995) J. Virol 69:5485-5496, Berns et al. (1996) Biology of Adeno-associated virus, in Adeno-associated virus (AAV) Vectors in Gene Therapy, K. I. Berns and C. Giraud, Springer (1996); and Chiorini et al. (1996) The Roles of AAV Rep Proteins in gene Expression and Targeted Integration, from Adeno-associated virus (AAV) Vectors in Gene Therapy, K. I. Berns and C. Giraud, Springer (1996)). A rep protein with improved activity could lead to increased amounts of virus progeny thus allowing higher productivity of rAAV vectors.

AAV and rAAV have many applications, including use as a gene transfer vector, for introducing heterologous nucleic acid into cells and for genetic therapy. Advances in the production of high-titer rAAV stocks to the transition to human clinical trials have been made, but improvement of rAAV production is complemented with special attention to clinical applications of rAAV vectors as successful gene therapy approach. Productivity of rAAV (i.e. the amount of vector particles that can be obtained per unitary manufacturing operation) is one of the rate limiting steps in the further development of rAAV as gene therapy vector. Methods for high throughput production and screening of rAAV have been developed (see, e.g., Drittanti et al. (2000) Gene Therapy 7:924-929) Briefly, as with the other steps in methods provided herein, the plasmid preparation, transfection, virus productivity and titer and biological activity assessment are intended to be performed in automatable high throughput format, such as in a 96 well (or other number or multiples thereof, such as 384, 1536 . . . 9600, 9984 . . . ) formats.

Since the Rep protein is involved in replication it can serve as a target for increasing viral production. Since it has a variety of functions and its role in replication is complex, it has heretofore been difficult to identify mutations that result in increase viral production. The methods herein, which rely on in vivo screening methods, permit optimization of its activities as assessed by increases in viral production. Provided herein are Rep proteins and viruses and viral vectors containing the mutated Rep proteins that provide such increase. The amino acid positions on the rep proteins that are relevant for rep proteins activities in terms of AAV or rAAV virus production are provided. Those amino acid positions are such that a change in the amino acid leads to a change in protein activity either to lower activity or increase activity. As shown herein, the alanine or amino acid scan revealed the amino acid positions important for such activity (i.e. hits). Subsequent mutations produced by systematically replacing the amino acids at the hit positions with the remaining 18 amino acids produced so-called “leads” that have amino acid changes and result in higher virus production. In this particular example, the method used included the following specific steps.

Amino Acid Scan

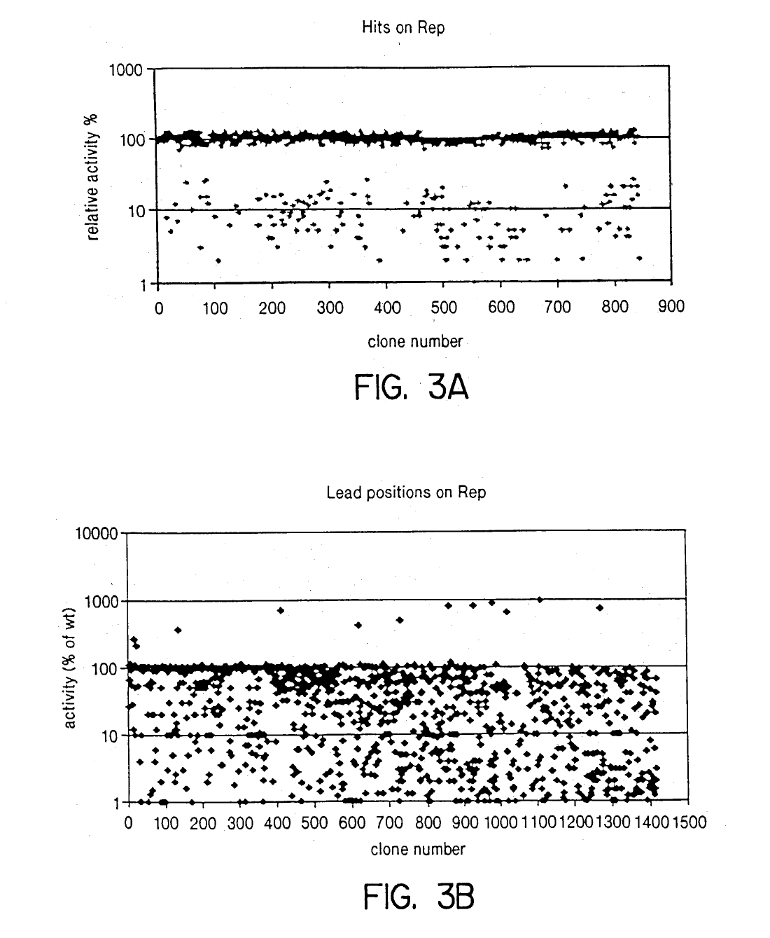

As discussed above, for nucleic acid modifications, a nucleic acid molecule scan is performed instead of an amino acid scan. In order to first identify those amino acid (aa) positions on the rep protein that are involved in rep protein activity, an Ala-scan was performed on the rep sequence. For this, each aa in the rep protein sequence was individually changed to Alanine. Each resulting mutant rep protein was then expressed and the amount of virus it could produced measured as indicated below. The relative activity of each individual mutant compared to the native protein is indicated in FIG. 3A. HITS are those mutants that produce a decrease in the activity of the protein (in the example: all the mutants with activities below about 20% of the native activity).

In a second experimental round, which included a new set of mutations and phenotypic analysis, each amino acid position hit by the Ala-scan step, was mutated by amino acid replacement of the native amino acid by the remaining 18 amino acids, using site directed-mutagenesis.

In both rounds, each mutant was individually designed, generated and processed separately, and optionally in parallel with the other mutants. Neither combinatorial generation of mutants nor mixtures thereof were used in any step of the method.

A plasmid library was thus generated in which each plasmid contained a different mutant bearing a different amino acid at a different hit position. Again, each resulting mutant rep protein was then expressed and the amount of virus it could produced measure as indicated below. The relative activity of each individual mutant compared to the native protein is indicated in FIG. 3B. LEADS are those mutants that lead to an increase in the activity of the protein (in the example: the ten mutants with activities higher, typically between 6 to 10 times, than the native activity).

Expression of the Genetic Variants and Phenotypic Characterization.

The rep protein acts as an intracellular protein through complex interaction with a molecular network composed by cellular proteins, DNA, AAV proteins and adenoviral proteins (note: some adenovirus proteins have to be present for the rep protein to work). The final outcome of the rep protein activity is the virus offspring composed by infectious rAAV particles. It can be expected that the activity of rep mutants would affect the titer of the rAAV virus coming out of the cells.

As the phenotypic characterization of the rep variants can only be accomplished by assaying its activity from inside mammalian cells, a mammalian cell-based expression system as well as a mammalian cell-based assay was used. The individual rep protein variants were expressed in human 293 HEK cells, by transfection of the individual plasmids constituting the diverse plasmid library. All necessary functions were provided as follows:

(a) the cellular proteins present in the permissive specific 293 HEK cells;

(b) the AAV necessary proteins and DNA were provided by co-transfection of the AAV cap gene as well as a rAAV plasmid vector providing the necessary signaling and substrate ITRs sequences;

(c) the adenovirus (AV) proteins were provided by co-transfection with a plasmid expressing all the AV helper functions.

A library of recombinant viruses with mutant rep encoding genes was generated. Each recombinant, upon introduction into a mammalian cell and expression resulted in production of rAAV infectious particles. The number of infectious particles produced by each recombinant was determined in order to assess the activity of the rep variant that had generated that amount of infectious particles.

The number of infectious particles produced was determined in a cell-based assay in which the activity of a reporter gene, in the exemplified embodiment, the bacterial lacZ gene, or virus replication (Real time PCR) was performed to quantitatively assess the number of viruses. The limiting dilution (titer) for each virus preparation (each coming from a different rep variant) was determined by serial dilution of the viruses produced, followed by infection of appropriate cells (293 HEK or HeLa rep/cap 32 cells) with each dilution for each virus and then by measurement of the activity of the reporter gene for each dilution of each virus. Hill plots (NAUTSCAN™) as described herein (published as International PCT application No. WO 01/44809 based on PCT No. PCT/FR00/03503, December, 2000; see EXAMPLES) or a second order polynomial function (Drittanti et al. (2000) Gene Ther. 7: 924-929) was used to analyze the readout data and to calculate the virus titers. Briefly, the titer was calculated from the second order polynomial function by non-linear regression fitting of the experimental data. The point where the polynomial curve reaches its minimum is considered to be the titer of the rAAV preparation. A computer program for calculation of titers has been developed (see Drittanti et al. (2000) Gene Ther. 7: 924-929) to assess the minimum.

The TREE method described herein can be used to analyze the readout data and to calculate the virus titers. The results are shown in the EXAMPLE below.

Comparison Between Results of Full-Length Hit Position Analysis Reporter Here and the Literature

The experiments identified a number of heretofore unknown mutation loci, which include the hits at positions: 4, 20, 22, 28, 32, 38, 39, 54, 59, 124, 125, 127, 132, 140, 161, 163, 193, 196, 197, 221, 228, 231, 234, 258, 260, 263, 264, 334, 335, 341, 342, 347, 350, 354, 363, 364, 367, 370, 376, 381, 389, 407, 411, 414, 420, 421, 422, 428, 429, 438, 440, 451, 460, 462, 484, 488, 495, 497, 498, 499, 503, 511, 512, 516, 517 and 518 with reference to the amino acids in Rep78 and Rep 68. Rep 78 is encoded by nucleotides 321-2,186; Rep 68 is encoded by nucleotides 321-1906 and 2228-2252; Rep 52 is encoded by nucleotides 993-2186, and Rep 40 is encoded by amino acids 993-1906 and 2228-2252 of wildtype AAV (see, FIGS. 5A and 5B).

Also among these are mutations that may have multiple effects. Since the Rep coding region is quite complex, some of the mutations may have several effects. Amino acids 542, 598, 600 and 601, which are in the Rep 68 and 40 intron region, are also in the coding region of Rep 78 and 52. Codon 630 is in the coding region of Rep 68 and 40 and non coding region of Rep 78 and 52.

Mutations at 10, 86, 101, 334 and 519 have been previously identified, and mutations, at loci 64, 74, 88, 175, 237, 250 and 429, but with different amino acid substitutions, have been previously reported. In all instances, however, the known mutations reportedly decrease the activity of Rep proteins. Among mutations described herein, are mutations that result in increases in the activity the Rep function as assessed by detecting increased AAV production.

Lead Identification.

Based on the results obtained from the assays described herein (i.e. titer of virus produced by each rep variant), each individual rep variant was assigned a specific activity. Those variant proteins displaying the highest titers were selected as leads and are used to produce rAAV.

In further steps, rAAV and Rep proteins that contain a plurality of mutations based on the hits (see Table in the EXAMPLES, listing the hits and lead sites), are produced to produce rAAV and Rep proteins that have activity that is further optimized. Examples of such proteins and AAV containing such proteins are described in the EXAMPLES.

The rAAV rep mutants are used as expression vectors, which, for example, can be used transiently for the production of recombinant AAV stocks. Alternatively, the recombinant plasmids may be used to generate stable packaging cell lines. To create a stable producer cell line, the recombinant vectors expressing the AAV with mutant rep genes, for example, are cotransfected into host cells with a plasmid expressing the neomycin phosphotransferase gene (neor) by transfection methods well known to those skilled in the art, followed by selection for G418 resistance.

Also among the uses of rAAV, particularly the high titer stocks produced herein, is gene therapy for the purpose of transferring genetic information into appropriate host cells for the management and correction of human diseases including inherited and acquired disorders such as cancer and AIDS. The rAAV can be administered to a patient at therapeutically effective doses. A therapeutically effective dose refers to that amount of the compound sufficient to result in amelioration of symptoms of disease.

The following examples are included for illustrative purposes only and are not intended to limit the scope of the invention. The specific methods exemplified can be practiced with other species. The examples are intended to exemplify generic processes.

### EXAMPLE 1

Materials and Methods

Cells:

293 human embryo kidney (HEK) cells, obtained from ATCC, were cultured in Dulbecco's modified Eagle's medium containing 4.5 g/l glucose (DMEM; GIBGO-BRL) 10% fetal bovine serum (FBS, Hyclone). Hela rep-cap 32 cells, described above, were obtained from Anna Salvetti (CHU, Nantes) and cultured in the medium described above.

Plasmids:

pNB-Adeno, which encodes the entire E2A and E4 regions and VA RNA I and II genes of Adenovirus type 5, was constructed by ligating into the polylinker of multiple cloning site of pBSII KS (+/−) (Stratagene, San Diego, USA) the SalI-HindIII fragment (9842-11555 nt) of Adenovirus type 5) and the BamHI-ClaI fragment (21563-35950) of pBR325. All fragments of adenovirus gene were obtained from the plasmid pBHG-10 (Microbix, Ontario, Canada). pNB-AAV encodes the genes rep and cap of AAV-2 was constructing by ligation of XbaI-XbaI PCR fragment containing the genome of AAV-2 from nucleotide 200 to 4480 into XbaI site of polylinker MCS of pBSIIKS(+/−). The PCR fragment was obtained from pAV1 (ATCC, USA). Plasmid pNB-AAV was derived from plasmid pVA1I, which contains the AAV genomic region, rep and cap. pNB-AAV does not contain the AAV ITR's present in pAV1. pAAV-CMV(nls)LacZ was provided by Dr Anna Salvetti (CHU, Nantes).

pCMV(nls)LacZ (rAAV vector plasmid) and pNB-Adeno were prepared on DH5a E. coli and purified by Nucleobond AX PC500 Kit (Macherey-Nagel), according to standard procedures. Plasmid pAAV-CMV(nls)LacZ is derived from plasmid psub201 by deleting the rep-cap region with SnaB I and replacing it with an expression cassette harboring the cytomegalovirus (CMV) immediate early promoter (407 bp), the nuclear localized β-galactosidase gene and the bovine growth hormone polyA signal (324 bp) (see, Chadeuf et al. (2000) J. Gene Med. 2:260-268. pAAV-CMV(nls)LacZ was provided by Dr Anna Salvetti.

Virus:

Wild type adenovirus (AV) type 5 stock, originally provided by Dr Philippe Moullier (CHU, Nantes), was produced accordingly to standard procedures.

rAAV Production:

3×10⁴ 293 HEK cells were seeded into each well of 96 microwell plates and cultured for 24 hours before transfection. Transfection was performing on about 70% confluent cells by 25 Kda PEI (poly-ethylene-imine, Sigma-Aldrich). Equi-molar amounts of AV helper plasmid (pNB-Adeno, 0.06 μg per well), rep-plasmid (wt rep or other rep mutant plasmid, 0.02 μg per well) and AAV vector plasmid (0.03 μg per well) and double amounts of cap-plasmid (0.04 μg per well) were mixed with 10 mM PEI (pH 7.4) by gently shaking using a ratio DNA/PEI=2.5 W/vol. The mixture was the added to the medium culture on the cells. 60 hours after transfection, the culture medium was replaced by 100 μl of lysis buffer. Serial dilutions of crude lysate were used to titer the rAAV productions and for biological activity testing of transgene.

For small particles of DNA/PEI complex formation: the PEI is added on DNA solution drop by drop. Mix at a maximum of 15 μl of complex PEI/DNA into 100 μl of production medium. Lysis buffer used depends upon the testing assay to be performed.

### EXAMPLE 2

Titering or Assessment of Concentration by a Method Designated Real Time Vector Titering (RTVT™)

This Example is based on the method described in International PCT application No. PCT/FR01/01366, based on French application No. 0005852, filed May 9, 2000, and published as International PCT application No. WO 01/186291. This method assesses the titer or concentration of a biological agent (virus, gene transfer vector) in a sample, by measurement of the kinetics of change of a reporter parameter following the exposure of cells to the biological agent.

As noted above, reporter parameters may include, but are not limited to: gene/transgene expression related to the gene/transgene products, such as enzymatic activity, fluorescence, luminescence, antigen activity, binding to receptors or antibodies, and regulation of gene expression), differential gene expression, viral/vector progeny productivity, toxicity, cytotoxicity, cell proliferation and/or differentiation activity, anti-viral activity, morphogenetic activity, pathogenetic activity, therapeutic activity, tumor suppressor activity, oncogenetic activity, pharmacological activity.

Serial Dilution Methods

The assessment of the concentration or titer of biological agents using current approaches needs for serial dilutions of the agent. Serial dilutions of the agent are applied to a cell-based reported system, that elicits an output signal in response to the exposure to the agent. The intensity of the signal is a function of the concentration of the agent. The titer or concentration of the agent is determined as the highest dilution that still elicits a measurable response in the output. The higher the number of dilutions tested, the higher the accuracy of the value obtained for the titer.

This approach requires a set of serial dilutions for every biological agent whose titer needs to be determined. Thus, the application of this approach to the simultaneous titration of a large number of different biological agents is limited by the number of experimental points needed (example: for 30 biological samples: 20 serial dilutions×30 biological agents: 600 experimental points).

The approach in International PCT application No. PCT/FR01/01366 published as International PCT application No. WO 01/186291

The intensity of the output signal (after exposure of reporter cells to the biological agent) is not only dependent on the concentration of the agent but also on the time after exposure. As time increases, the intensity of the signal increases. The kinetics of change of the intensity over time depends upon the concentration of the agent. Thus, lower concentrations of the agent require longer times for the intensity to reach a given value that would be reached in shorter times after exposure to higher concentrations of the same agent.

This approach (designated Real Time Virus Titering (RTVT™) uses the following: a reference plot representing the relationship between the concentration of the agent and the time necessary for the intensity to reach a given threshold value is obtained using a reference preparation of biological agent, whose concentration or titer is known. This plot is then used to obtain the concentration of the biological agent under study by entering the time that a dilution of that agent needed for the intensity to reach the threshold value.

Using this approach, there is no need for serial dilutions of the biological agent(s) under study. Once the reference plot (tβ vs c) is obtained, it can be used for the determination of the concentration or titer of any number of biological agents. Only one dilution of the biological agent under study is necessary to obtain the corresponding value of tβ that is then used to obtain the concentration or titer using the reference plot.

Thus, the application of this approach to the simultaneous titration of large numbers of different biological agents is facilitated by the fact that only one dilution of each sample is needed (example: for 30 biological samples: 1 dilution×30 biological agents: 30 experimental points (compared to 600 needed with the current approach). This approach is specially suited for the high throughput assessment of concentration or titer of large numbers of biological agents.

The system

The system includes the following elements:

a preparation of the biological agent (virus, gene transfer vector, protein, . . . ) whose concentration or titer is unknown and has to be determined.

a reporter system including culture of a cell line (or a mixture of cell lines) that reacts to the exposure to the biological agent by displaying a specific output signal.

a master preparation of a reference biological agent, of known concentration or titer, that is able to generate the output signal when the reporter cells are exposed to it.

Practice of the Method

When the reporter cells are exposed to either the biological agent under study or the reference biological agent, an output signal is generated, that can be measured.

The intensity of the output signal is called i; the concentration of the biological agent used is called c, the time of exposure of the cells to the biological agent is called t. The intensity of the output signal (i) is a function of c and t:

i increases as the concentration (c) of the biological agent applied to the cells increases;

i increases as the time of exposure of the cells to the biological agent (t) increases.

If the time t after exposure of the cells to the biological agent is kept constant, then, i changes as a direct function of c.

If the concentration c of the biological agent is kept constant, then, i is a direct function of t.

β is defined here as a threshold value of the intensity of the output signal, arbitrarily defined for every system under study.

tβ is defined here as the time necessary to reach the threshold β.

Use of β and tβ to Determine the Concentration or Titer of a Biological Agent.

The reporter cells are exposed to serial dilutions of a reference biological agent, whose concentration (or titer) is previously known. The intensity of the output signal (i) is measured at several time points (t) for every concentration (serial dilutions) c of the reference biological agent.

i is plotted vs t, and that, for every concentration c used of the reference biological agent.

Using the plots obtained above, and for every concentration c of the reference biological agent, the time (tβ) necessary for the intensity of the output signal to reach a threshold value β is obtained.

With the data obtained above, tβ is plotted vs c.

This plot represents the time necessary for the intensity of the output signal to reach the threshold value β as a function of the concentration of the biological agent used. This is a standard plot and is used to determine the unknown concentration of the biological agent under study by measuring the time that a given dilution of it needs to give an output signal whose intensity equals the threshold β. The reporter cells are exposed to a dilution of the biological agent under study (whose concentration or titer is to be determined). The intensity of the output signal (i) is measured over time until it reaches the threshold value β. The time necessary for i to reach the value β is recorded as tβ.

The tβ value recorded above is entered into the standard plot obtained above) and the corresponding c value is obtained.

This c value represents the concentration or titer of the biological agent under study.

Example of the Real Time Virus Titerng RTVT™ Titering Method

Rat-2 cells were infected with serial dilutions of a reference preparation of a retroviral vector carrying the green fluorescent protein (GFP) gene (vector pSI-EGFPI see, Ropp et al. (1995) Cytometry 21:309-317). At increasing times after infection, the level of expression of the transgene was determined (as the level of fluorescence due to the GFP gene) as the output signal.

Table 3 represents the values obtained:

| Concentration | Time after | Output signal |
|---|---|---|
| (1 = 10⁶ particles/ml) | infection (hrs) | fluorescence |
| 0.1 | 16 | 20.4 |
| | 24 | 30.1 |
| | 40 | 95.1 |
| | 48 | 138.7 |
| | 64 | 157.3 |
| 0.25 | 16 | 26.8 |
| | 24 | 48.5 |
| | 40 | 173.3 |
| | 48 | 228.2 |
| | 64 | 191.7 |
| 0.5 | 16 | 38.1 |
| | 24 | 72 |
| | 40 | 198.7 |
| | 48 | 296.2 |
| | 64 | 203.7 |

The threshold value of β=100 was arbitrarily selected for this example. The time (tβ) necessary for the output signal to reach the threshold β, for every concentration is shown in table 4.

TABLE 4

| Concentration | tβ |
|---|---|
| (1 = 10⁶ particles/ml) | (hrs) |
| 0.1 | 42 |
| 0.25 | 31 |

A plot of tβ versus concentration for the reference virus shows that the concentration and tβ exhibit a clearly defined relationship, that allows for the calculation of the concentration (c) of a sample, if the corresponding tβ of that sample is known.

### EXAMPLE 3

Tagged Replication and Expression Enhancement (TREE) for Titering

As discussed above, TREE is a method for titering and standardization of preparations of viruses, vectors, antibodies, libraries, proteins, genes and any other moiety that is detectable based upon a output signal, such as fluorescence. The TREE method is an improvement of the Real Time Virus Titering (RTVT) method (see, International PCT application No. PCT/FR01/01366 published as International PCT application No. WO 01/186291). It is performed a reporter moiety, such as a reporter virus (with a known titer) and the test sample (with unknown titer). The reporter, such as a reporter virus has a readily detectable output signal that can be measured as a function of time. The effect of the moiety, such as a virus, of unknown titer is assessed. The moiety whose titer is assessed either increases or decreases the output signal as a function of time. This change in signal is used to assess the amount or concentration of the moiety of unknown concentration, and hence its titer.

The method is exemplified herein using an AAV system for the determination of the titer of an AAV vector and an AAV-reporter vector as a competitor and wild type Adenovirus as helper virus. One of skill in the art readily can adapt the method to other systems, including other viruses, and other moieties for which a reporter system can be developed. Other such moieties include, but are not limited to, viral vectors, plasmids, libraries, proteins, antibodies, vaccines, genes, and nucleic acid molecules.

Materials and Methods

1. Cells and Viruses

HeLa rep-cap32 cells, a HeLa derived cell line (kindly provided by P. Moullier, Laboratoire de Thérapie Génique, CHU, Nantes; see, Salvetti et al. (1998) Hum Gene Ther 20:695-706; Chadeuf et al. (2000) J Gene Med 2:260-268) was grown in DMEM with 10% fetal calf serum. These cells were plated 24 h before infection at a density of 1×10⁴ cells in single well of 96-well plates. rAAV-LacZ (10¹⁰ ip/ml), rAAV-GFP (10⁹ ip/ml) vectors and Human Adenovirus type 5 (Ad5) (10¹¹ pfu/ml) were from CHU, Nantes.

Hela rep-cap32 cells had been produced by cotransfecting plasmid pspRC, which harbors the AAV rep-cap genome with the ITRs deleted (bp 190 to 4484 of wild-type AAV), with plasmid PGK-Neo, conferring resistance to G418 on Hela cells (see, Chadeuf et al. (2000) J. Gene Med. 2:260-268 and Salvetti et al. (1998(Hum Gene Ther. 9:695-706). Hela rep-cap 32 cells are a packaging line that harbor one copy of the genome with the ITRs deleted (see, also Tessier et al. (2001) J. Virol. 75:375-383).

Plasmid pspRC contains the AAV genome (positions 190-4,484 bp) with the ITRs deleted and was obtained by excising the rep-cap fragment (XbaI fragment) from the well-known vector psub201 (Samulski et al. (1987) J Virol 61:3096-3101; also called pSSV9) by XbaI digestion and inserting it into the XbaI site of plasmid pSP72 (Promega). Plasmid psub201 (see, e.g., U.S. Pat. No. 5,753,500) is a modified full-length AAV type 2 genomic clone contains all of the AAV type 2 wild-type coding regions and cis acting terminal repeats.

2. Infection and Measurement

Four serial dilutions of a rAAV-LacZ (0.01, 0.0075, 0.005 and 0.0025 μl, see Table 2 below, designated samples 1-4, respectively) were made and used for co-infection of HeLa rep-cap32 cells together with 8 different Ad5 multiplicity of infection (MOI; from 0.1 to 100/cell) and with 10⁻³ ml (10⁶ infectious particles (ip)) or 10⁻⁴ ml (10⁵ ip) rAAV-GFP viral vector. All the samples were done in triplicate. After infection, the plates were read at different times, from 34.5 h to 80 h (every 30 minutes).

rAAV-GFP is an SSV9-derived vector; SSV9 is a clone containing the entire adeno-associated virus (AAV) genome inserted into the PvuII site of plasmid pEMBL (see, Du et al. (1996) Gene Ther 3:254-261). The rAAV-GFP and rAAV-LacZ plasmids are SSV9 with a GFP or LacZ gene under control of the cytomegalovirus (CMV) immediate-early promoter. All the samples were done in triplicate.

3. Process

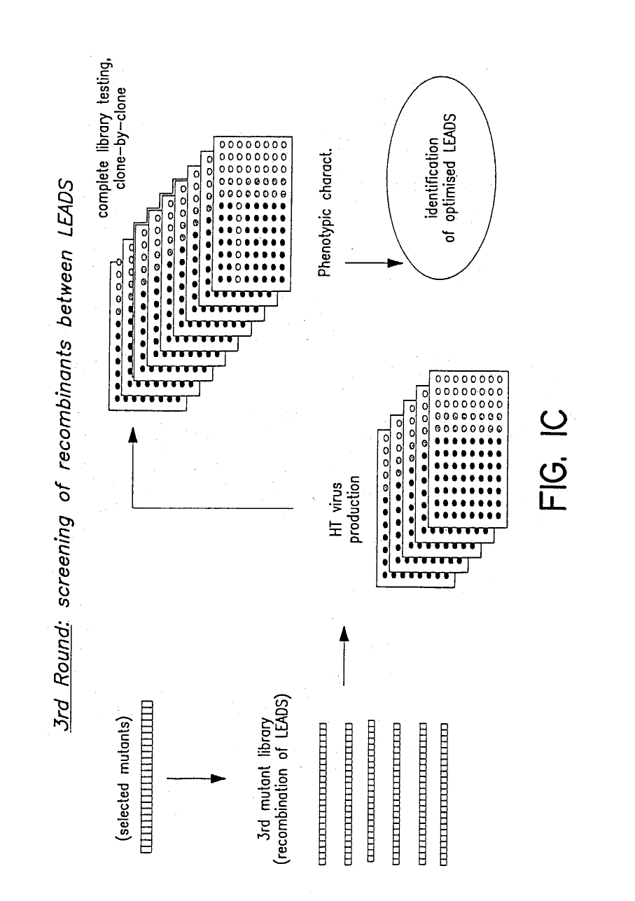

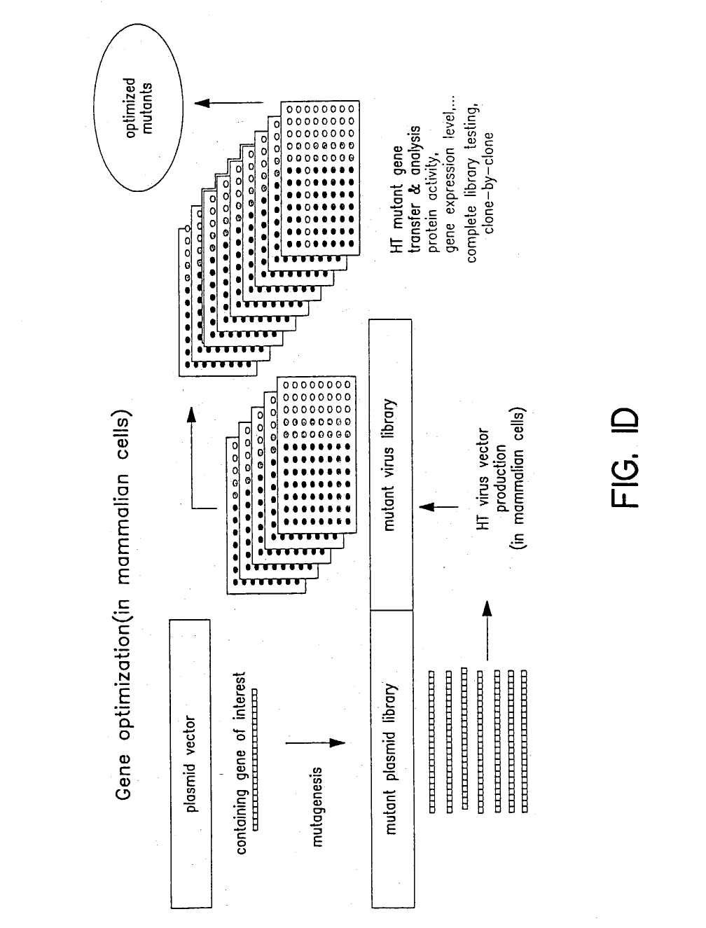

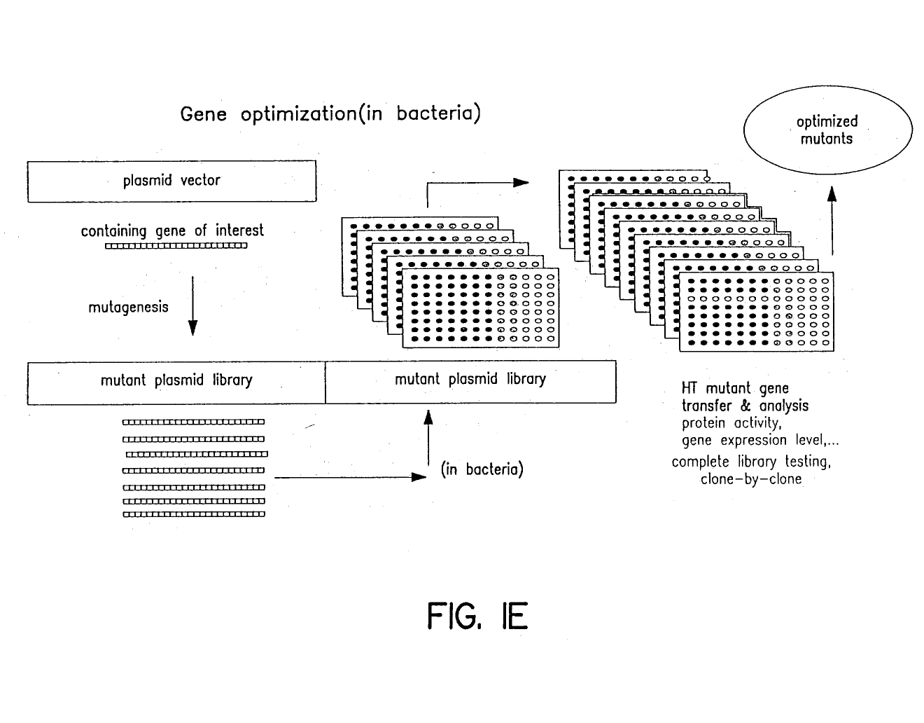

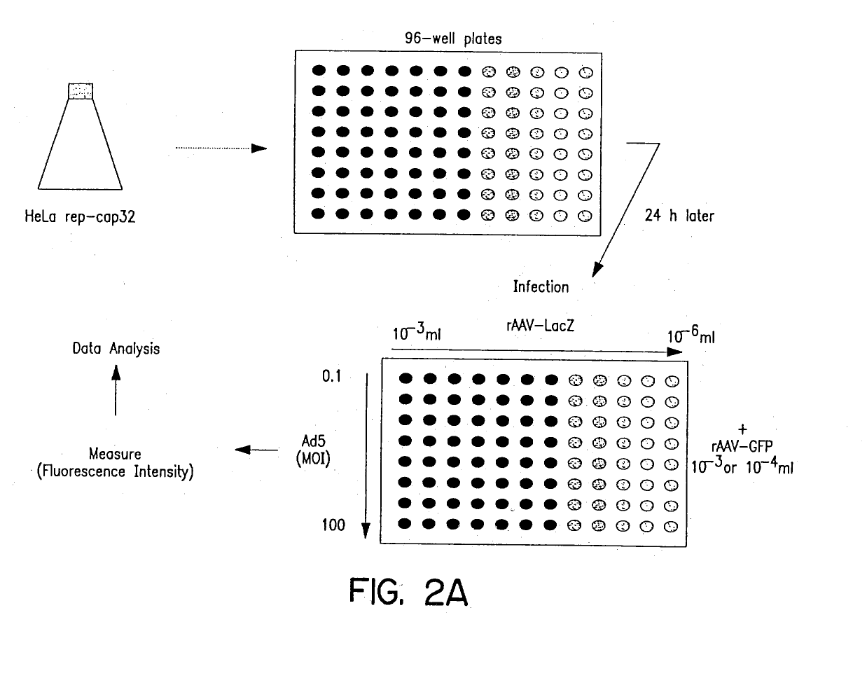

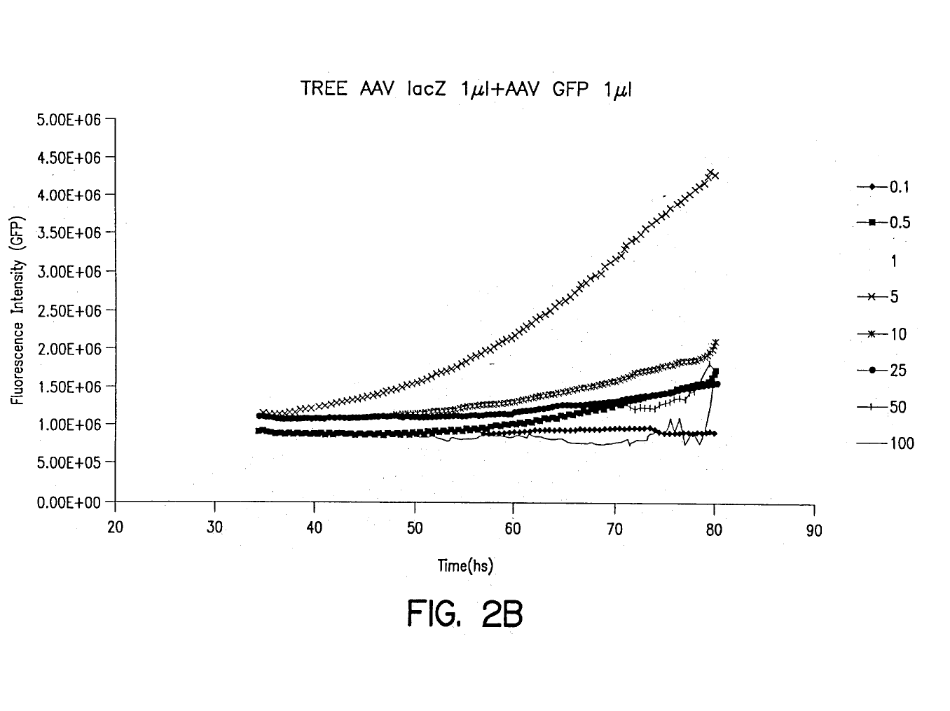

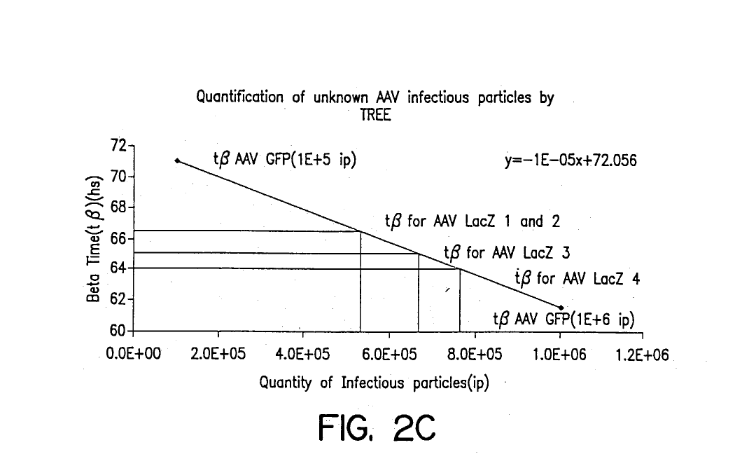

FIG. 2 shows the overall procedure in 96 well format. Cells were plated 24 h before infection. Co-infection of rAAV-GFP with serial dilutions of rAAV-LacZ together with Ad5 (different MOI), were done. Then the plates were read at different times using the Analyst AD&HT micro plate-reader (LJL BioSystems).

4. Analysis

For this kinetic technique, Fluorescence Intensity (FI) of the infected cells is measured as a function of the time. Serial dilutions of the AAV-competitor vector AAV-lacZ vector, which decreases the fluorescence signal, are performed. For this example, fluorescence was measured for AAV-GFP with 10⁶ ip and 10⁵ ip and then 10⁶ ip of the AAV-GFP reporter virus in the serial dilutions of the competitor virus, AAV-lacZ vector in a 96-well format (samples 1-4, see Table below). Measurements were taken of each well and curves of FI (of the GFP) versus time (hrs) were obtained (see FIG. 2B).

An arbitrary one value for FI (see FIG. 2B, 6×10⁶ FI units), typically, though not necessarily, near the greatest separation among the curves so that the numbers are readily discernable, was selected. The point at which each of the curves intersect this value is beta time (tβ) for that combination of amounts of reporter plus dilution of the virus of unknown titer. tβ, taken from the FI vs. Time (hrs) curves, for each sample containing a dilution of the unknown plus 10⁶ ip of the reporter virus is set forth in column 2 of Table 2 below.

To determine the titer of the test virus, the tβ for the AAV GFP (reporter virus) is plotted versus quantity of ip (i.e a straight line between the tβ for the 10⁶ ip and the 10⁵ ip) (FIG. 2C). For any tβ of the unknown virus, the quantity of ip can be determined from this curve. The beta time (tβ) of each sample (in this case for the different dilutions of rAAV LacZ mixed with 10⁶ infectious particles of rAAV-GFP) is determined, and then the residual number of infectious particles of rAAV-GFP for each sample. The difference between 10⁶ ip of rAAV-GFP put in each sample and the number of ip detected by fluorescence in the same well is the actual quantity of rAAV-GFP competed (consumed) by the unknown rAAV (in this case rAAV-LacZ). This number is determined for each dilution. The quantity rAAV-GFP consumed is the same quantity of unknown rAAV in the sample. This quantity is present in one volume of unknown rAAV, which in this example is 1 ml. Based upon this, the infectious titer of the unknown rAAV is determined. The results are shown in Table 2.

TABLE 2
AAV LacZ titration by TREE titration

| | | | | | AAV LacZ |
|---|---|---|---|---|---|
| | | | Residual | Consumed | Concentration |
| Sample | volume (μl) | tβ (hrs) | AAV-GFP | AAV-GFP | (i.p./10⁻² μl) |
| 1 | 10.0 × 10⁻³ | 66.5 | 5.56 × 10⁵ | 4.44 × 10⁵ | 4.44 × 10⁵ |
| 2 | 7.5 × 10⁻³ | 66.5 | 5.56 × 10⁵ | 4.44 × 10⁵ | 5.93 × 10⁵ |
| 3 | 5.0 × 10⁻³ | 65 | 7.06 × 10⁵ | 2.94 × 10⁵ | 5.88 × 10⁵ |
| 4 | 2.5 × 10⁻³ | 64 | 8.06 × 10⁵ | 1.94 × 10⁵ | 7.76 × 10⁵ |

The average titer using this method was 6.01 × 10¹⁰ ip/ml (6.01 × 10⁷ ip/μl = 6.01 × 10⁵ ip/0.01 [2l). The standard deviation was 1.37 × 10¹⁰ ip/ml with an error of ±23%.

### EXAMPLE 4

Hill Analysis of the Screening Assay Output

It is important to have reliable methods for screening and/or evaluating the performance of a set of biological agents, such as a library of viral or non-viral recombinant vectors, vaccines, recombinant proteins and antibodies, in a complex biological system, such as living target cells When developing such agents, for example gene therapy vectors and other agents for therapeutic use, it is necessary to be able to evaluate and compare performance among candidates.

The progress of gene transfer into gene therapy depends upon the capacity to develop gene transfer vectors into therapeutic drugs. Clinically relevant vectors need to be efficient and safe, in reaching and infecting target cells and in ensuring a persistent level of expression of the therapeutic gene with a minimum of adverse effects. The availability of standardized quantitative methods, suitable for an accurate and objective assessment of titer, performance and safety, is necessary for the pharmaceutical development of gene vectors as drugs.

Any method for assessment is contemplated herein as long as it is adapted for use in a high throughput format. Of particular interest is the Hill equation based method of International PCT application No. WO 01/44809 (International PCT application No. PCT/FR00/03503, based on French application FR 9915884).

Two widely used parameters that provide quantitative information about the potential performance of a gene transfer vector preparation are the titer of physical particles and the titer of infectious particles. Vector preparations with high titer of infectious particles and low physical particles/infectious particles ratio are considered to be of higher quality.

The titer in physical particles(pp) (see, e.g., Mittereder et al. (1996) J. Virol. 70:7498-7509; Atkinson et al. (1998) Nucl. Acids Res. 26:2821-2823; Kechli et al. (1998) Hum. Gene Ther. 9:587-590; and Nelson et al. (1998) Hum. Gene Ther. 9:2401-2405), which represents the total number of vector particles, is usually evaluated from the vector content by detecting the nucleic acid contents (nucleic acids hybridization and OD₂₆₀ respectively for AAV and AdV), detecting viral protein content (for example, reverse transcriptase (RT) activity and p24 content for MLV and HIV, respectively).

Among the physical particles (pp), there are particles potentially active in performing transduction (ip, infectious particles), as well as particles that are inactive (nip, non-infectious particles) (Ruffing et al. (1994) J. Gen. Virol. 75:3385-3392; Kechi et al. (1998) Hum. Gene Ther. 9:587-590.). The pp and the ip/nip ratio, are features of the packaging system, the manufacturing process and the vector itself.

The infectious particles (ip) (infectious units, transducing units, etc.) are evidenced by the changes observed in the infected cells (vector DNA replication, provirus integration, cell lysis, transgene expression and other observable parameters. Infectious particles (ip) measures the number of particles effective in performing a process whose output is being measured; not all particles participate or are capable of participating in all processes.

The precise assessment of ip is not straightforward. Existing methods are mainly based on serial dilution experiments followed by either linear extrapolation or asymptote approximates. The titer of infectious particles (ip: infectious unity, transduction unity) (see, e.g., Mittereder et al. (1996) J. Virol. 70:7498-7509; Weitzman et al. (1996) J. Virol. 70:1845-1854; Salvetti et al. (1998) Hum. Gene Ther. 9:695-706) is evaluated by the studying observed changes in infected cells, such as viral replication, provirus integration, cellular lysis and transgene expression, using methods based on serial dilutions, followed either by a linear extrapolation or an asymptotic approximation. Thus, ip measures the number of active particles in the measured process; it includes physical particle (pp) and inactive particles (nip or non-infectious particles).

In order to resolve the problem of the titer determination and the comparison of different recombinant viruses used in gene therapy, the variation of the particles/infectious power ratio has been used (see, e.g., Atkinson et al. (1998) Nucl. Acids Res. 26:2821-2823; and International PCT application No. WO 99/11764, which describe a method that uses step of amplification viral genetic material in a host cellular line, preparation of vectors of unknown titer obtained by serial dilution and an internal check of known titer). In particular, the method uses cells infected with a viral preparation in the different wells of a microtiter plate, viral genome replication in the host cells, nucleic acid hybridization and determination of the relative amount of replicated viral nucleic acid in each well.

All of these methods measure the physical particles (pp) titer and/or measure infectious particles (ip) titer in order to evaluate a gene transfer vector. A high quality vector preparation is one with an high titer of infectious particles and a low pp/ip ratio. These parameters provide quantitative information on the performance of a gene transfer vector. Because of the inaccuracy of the procedures used for assessing pp and especially ip, these parameters are not informative enough to precisely define the features of a gene therapy vector nor those of a particular preparation thereof. The actual procedures used for pp and ip evaluation change with the vector type, are not very reproducible nor exact, so these parameters do not contain enough information to allow a very fine definition of vector characteristics and performances.

Hill Equation-Based Analyses

In this method complex biological processes, including those involving the response of cells (in vitro and in vivo) to biological agents, such as, for example, cells, viruses, vaccines, viral and non-viral gene vectors, antibodies, antigens, proteins in general and plasmids, are characterized using the formal analysis first introduced by Hill (see, Hill (1910) J. Physiol. 40:4P; Hill (1913) I. Biochem.J. 7:471-480). International PCT application No. WO 01/44809 (based on PCT/FR00/03503, priority claimed to French application FR 9915884) describes the use of the Hill equation (see, Hill (1910) J. Physiol. 40:4P; Hill (1913) I. Biochem.J. 7:471-480; see, International PCT application No. PCT/FR00/03503) for analysis and characterization of the biological and/or pharmacological activity of biological agents (viruses, vectors or cells) on biological assay systems in vitro (cell-based) or in vivo.

A number of useful parameters, derived from the Hill equation, are scored and used to quantify relevant features of the biological agent, of the cells, as well as of the biological process or reaction involved.

In particular, methods for calculation and analysis of the parameters of biological and pharmacological activity of native, attenuated, modified or recombinant viruses, vaccines, recombinant viral and non-viral gene transfer vectors, cells, antibodies and protein factors in in vitro (cell-based) or in vivo assays are described. This method is adapted for high throughput processes and is sufficiently accurate to allow a very fine definition of vector characteristics and performances.

International PCT application No. WO 01/44809 provides, a standard process for evaluating the interaction between any biological agent, such as a gene therapy vector, with a complex biological system (living target cells). It provides a screening process for a pool of complex biological agents, in order to select test agents that have a desired property, activity, structure or whatever is being sought.

Different biological agents and assay systems (cells) are compared and ranked out on the basis of their performance, assessed through the Hill parameters. Thus, the accurate analysis and comparison of the biological response of complex assay systems (in vitro and in vivo) to complex biological agents is achieved experimentally. The Hill-based analysis (π,κ,τ,ε,η,θ) is used for a variety of purposes, including, but not limited to:

i) validation and optimization of the manufacturing processes used to obtain the biological agents;

ii) development and optimization of the components of the biological agents (proteins, genomes, genetic units);

iii) development and optimization of assays and analytical tests for the characterization of the biological agents.

The method includes the steps of:

(a) preparation, for each biological agent, of a sample scale, obtained by a serial dilution of the biological agent at a R1 concentration,

(b) incubation of each sample of the dilution scale obtained in 1, with the target cells at a constant concentration R2,

(c) determination of the P product from the reaction R1+R2, at a t moment, in each the sample; and

(d) realization of a theoretical curve H from the experimental points R1 and P, for each biological agent by iterative approximation of parameters of the reaction R1+R2→P, at the t moment, in accordance with this equation:

P=Pₘₐₓ(πR1)^{r}/(κ+(πR1)^{r}) r=1, . . . ,n (2)

in which:

R1 represents the biological agent concentration in a sample from the scale;

R2 is concentration of target cells (in vitro or in vivo)

P (output) represents the product from the reaction R1+R2 at a t moment;

Pₘₐₓ represents the reaction maximal capacity;

κ represents, at a constant R2 concentration, the resistance of the biological system for responding to the biological agent (resistance constant R2);

r represents a coefficient that depends on R1 and corresponds to the Hill coefficient; and

πrepresents the intrinsic power of the R1 biological agent to induce a response in the biological system (P production at the t moment), and

(e) sorting the κ and π values obtained in (d) for each biological agent and the biological agent, and then ranking according to the values thereof.

Using the parameters (π,κ,τ,θ,ε,η) the activity of a biological agent on a complex biological system, as well as its intrinsic features can be fully characterized and compared. In addition, different biological systems either in vitro cell-based) or in vivo can be compared.

Hill Equation

The Hill equation:

$$P = {\sum }_{r = 1}^{r = n} {P}_{max} \cdot {R1}^{r} / ( K + {( R1 )}^{r} ) R2 constant \qquad ( 1 )$$

where R1, P, Pₘₐₓ and K represent, respectively, the concentration of the reagent R1, the concentration of the product, the maximal capacity of the reaction and the ‘affinity’ constant between R1 and R2. The Hill's coefficient (r) is a function of R1. The coefficient r is equal to 1 when independent non-interactive binding sites are involved between R1 and R2, such as in reactions that follow kinetics described by Michaelis-Menten; and r varies from 1 to n for systems where the sites involved in the interaction between the R1 and R2 are not independent from each other, and the affinity for R1 at any R2 binding site varies as a function of either i) the degree of occupancy of other R2 sites; ii) the concentration of R1 itself or iii) the concentration of other (positive or negative) regulators.

The Hill equation, thus, is a general formalization that describes the interaction reaction between molecules. It expresses the amount of product formed as a function of the concentration of the reagents and of the affinity constant of the system. Originally developed for the study of the dissociation between haemoglobin and oxygen, the Hill equation covers the formal Michaelis-Menten analysis of enzyme kinetics, the analysis of ligand-receptor binding and of the allosteric protein systems.

According to Hill, for a simple reaction like

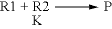

where the affinity between R1 and R2 changes with concentration of each, the Hill equation describes the accumulation of the product P as a function of the concentration of one of the reagents (R1) and of the intrinsic properties (K) of the system.

This equation can be applied to complex biological systems. For example, the response of the cells to infection (P), can be analyzed by applying an Hill-type equation. The amount of cells growing in vitro (R2) are infected with increasing concentrations of recombinant viruses (R1), and (P) is monitored. A Hill equation is iteratively fitted to the experimental data.

For analyses of viral output as exemplified herein,

←______κ______→

virus+cell transduced cell output (viral genome replication), Equation (1) is specifically reformulated as:

P=Pₘₐₓ(πR1)^{r}/(κ+(πR1)^{r}) r=1, . . . ,n (2)

where P, Pₘₐₓ, R1, π, r and κ, as described above, represent, respectively, the output signal (P) (the level of viral gene expression, or the level of virus replication), the maximal output signal (Pmax), the initial concentration (R1) of infectious viral particles (those susceptible to trigger the process leading to P), the potency of the vector (π; a factor that affects the concentration of the vector (R1) by its specific strength or activity, the Hill's coefficient (r) and the constant of resistance of the reaction or process (κ).

The Constant of Resistance κ

The concept of κ is analogous to those of dissociation, kinetics, equilibrium or affinity constants concepts for simple chemical and biological reactions. κ is a feature of the process (reaction) and of the biological system tested (cell type). κ is a key parameter for the characterization of the assay system and the assessment of its performance as a test for the reaction under study.

κ measures the internal resistance offered by the process or reaction triggered by the biological agent, to proceed to P. κ is specific to a particular process or reaction tested. In addition κ is specific to the particular biological system tested. Different cell lines and types displays different κ for the same reaction. Moreover, factors affecting the performance of a cell to accomplish the reaction (like contaminants, toxic agents, etc.) affect κ in that cell.

Variations in κ affect equation (2) by shifting the curve to the right or to the left, according to whether the value of κ increases or decreases, respectively. All curves differing only in κ are parallel each other. κ finds its direct and practical application in i) assay development and validation and ii) assessment of the susceptibility or sensitivity of different cell types or tissues to undertake the reaction under study and to be affected by it.

The potency π

πmeasures the intrinsic potency of the biological agent to accomplish P against the resistance (κ) offered by the reaction process. For every infectious virus particle (R1) added to the assay, the actual activity of the virus added is given by πR1. In order to report an output P, the potency π has to push forward the reaction inside the cell against κ. π is specific to the particular biological agent for the reaction under study. π is a feature of the biological agent.

Different versions or variants of the biological agent displays different π for the same reaction. Thus, mutations, conformational changes or other modifications on the biological agent are expected to change its π for a given reaction process.

The concept of π is analogous to that of chemical activity by opposite to concentration for simple compounds. π is a correction factor that affects the concentration (R1) of the biological agent to indicate its actual strength or activity for a given reaction process.

Variations in π affect equation (2) by shifting the curve to the right or to the left, according to whether the value of π decreases or increases, respectively. Curves differing only in π are not parallel each other. The slope of the curve given by equation (2) increases as π increases.

π is a key parameter for the characterization of the biological agent and the assessment of its performance to accomplish the reaction under study. π finds its direct and practical application in i) biological agent optimization and development as it allows to compare the relative potency of variants of the agent.

π is a valuable tool in the field of vaccine, gene transfer vector and antibody development, for the comparison between two or more different agents or different versions of the same agent, for performance. Two agents, for instance, may elicit equivalent potencies for gene transfer, while their potencies for immunogenicity be different. The use of π, a quantitative and accurate parameter for assessing potency, permits ranking of the candidates according to their potency (i.e., for gene transfer, gene expression, immunogenicity and other such properties and activities) and to make rational decisions about the relative value of the agent leads.

The Efficiency ε

ε measures the maximal global efficiency of the reaction process when a biological agent characterized by a given π value interacts with a biological system characterized by a given κ. ε is specific to the particular couple biological agent (π)/biological system (κ) for the reaction under study. ε is a feature of the global reaction process and intervening reagents. Changes in either π, κ, or both, lead to changes in e.

The efficiency of the reaction process described by equation (2) is given by the increase in the output P that can be obtained by increasing the input R1. Thus, the first derivative of P with respect to R1, or the slope of the curve described by equation (2), gives the global efficiency of the reaction at every R1 input. The maximal global efficiency, or ε, is given directly by either the slope at the inflection point of the curve described by equation (2) or by the maximum of its derivative δP/δR1. The slope of the curve given by equation (2) and the maximum of δP/δR1 increase as ε increases.

ε is a key parameter for the characterization of the efficiency global process, considering the assay conditions and reagents all together. It is therefore useful for assay optimization once π and κ have been fixed and to detect changes in π when κ is kept constant or, inversely, changes in κ while π is kept unchanged.

The Heterogeneity Index η

η measures the internal heterogeneity of the reaction process under study. Complex processes include a huge chain of individual and causally events inside a multidimensional network of interrelated and interregulated biological reactions. Thus, the constant of resistance (K) for the particular reaction process under study is a macroscopic indicator of the global resistance of that process (K=a1κ1+a2κ2+ . . . anκn/n). If the contribution of the individual microscopic constants of resistance (a1κ1, a2κ2, . . . anκn) for the individual steps involved in the process were homogeneous and no thresholds were present from one step to the next, then, no discontinuities in the increase of the Hill coefficient (i.e. in the change of K) with R1 should be observed. The existence of a major heterogeneity among the κi values corresponding to the microscopic individual steps (i.e. the existence of thresholds for the intermediate steps) might lead to a macroscopic discontinuity in the system. Heterogeneity would cause a change in the rate of variation of the Hill coefficient and, which would require a jump in the macroscopic value of κ in order for equation (2) to fit the data.

The presence of internal heterogeneity in the reaction process can be detected by the appearance of steps in the rate of change of the Hill coefficient, corresponding to the Hill curve that fits the experimental data. η_is defined as an index of heterogeneity and its value corresponds to the number of steps in the rate of variation of the Hill coefficient (one step, κ=1; two steps, η=2; n steps, η=n).

ηis a key parameter for the dissection and detailed analysis of the reaction process. It is useful for the independent optimization and development of every one of the steps identified by η.

As mentioned, the presence of steps in the rate of change of η translates in an abrupt discontinuity in κ. Therefore, every step is determined by a different macroscopic constant of resistance κ. Systems with η=2, can thus be described by a Hill equation in which κ takes two different values (κ1 and κ2), according to the R1 interval considered. One part of the curve is described by κ1 and the other by κ2.

Hill curves describing reaction processes characterized by η=2, are hybrids generated from two parallel Hill curves differing only in κ. The transition from one curve to the other may alter the smooth change in the slope of the resulting Hill curve.

The Apparent Titer τ

In the Hill equation (2), when R1 increases, r increases from 1 to 2,3,4 . . . and P approaches its Pₘₐₓ value. On the other direction, on the contrary, R1 can only decrease up to a minimal point (R1ₘᵢₙ), at which r and P reach their minimal values. The Hill sigmoidal curve is not symmetric, only the right arm is asymptotic (towards Pₘₐₓ). On the left arm, the curve has an origin at R1ₘᵢₙ; the empirical curve does not fit the data for values below R1ₘᵢₙ.

From a biological point of view, the fact that P does not exist for R1 below R1ₘᵢₙ, means that there is no ‘product’ when the concentration of ‘substrate’ is lower than R1ₘᵢₙ; e.g. that the system is not responsive to concentrations below R1ₘᵢₙ. The minimal concentration of R1 that the system can detect and report is R1ₘᵢₙ.

In terms of biological agents, R1ₘᵢₙ represents the minimal amount that can elicit a response in a given reporter system, and it is represented by τ. The titer defined this way, is neither an asymptote value nor a value approached by extrapolation, but a precise parameter of the Hill equation, at the very mathematical origin of the curve.

τ measures the limiting dilution or apparent titer of the biological reagent. The value of τ is determined by the limit of sensitivity of the biological assay system and of method used for the measurement of the product P; that is why it is said to be apparent titer.

τ is specific to the batch or stock of the biological reagent tested. τ represents the apparent concentration of the biological agent and is expressed in units per volume, e.g. the maximal dilution of the biological agent that leads to the production of P. τ is given by the maximal R1 for which the Hill coefficient reaches its minimal value (the Hill coefficient becomes constant at a value equal or close to 1). The concept of τ corresponds to that of titer, of general use for viruses, antibodies and vectors. Variations in τ affect equation (2) by shifting the curve to the right or to the left, according to whether the value of τ decreases or increases, respectively.

τ is a key parameter that measures the ‘apparent’ concentration of a stock of the biological agent, which is necessary for whatever use given.

The Absolute Titer θ

θis the parameter that measures the absolute concentration (titer) of a stock or batch of the biological agent. The value of θ is not determined by nor dependent on the limit of sensitivity of the biological assay system or of the method used for the measurement of the product P; that is why it is said to be absolute titer. θ is specific to the batch or stock of the biological reagent tested. It represents the real physical concentration of the biological agent and is expressed in units per volume, e.g. the maximal dilution of the biological agent that leads to the production of P.

θ is given by the following equation

θπ=τ/s (3),

where s is the sensitivity of the detection method. Therefore, for agents detected using the same method, the following expression is valid:

θ1π1/τ1=θ2π2/τ2=θnπn/τn=constant (4)

Using equation (4), the ratio of the absolute titer θ, corresponding to two biological agent preparations, can be obtained from their respective π and τ. Variations in θ affect the equation (2) by shifting the curve to the right or to the left and/or by changing its slope.

Compensation Between π and κ

π and κ may appear to compensate to generate two different Hill curves (one differing in π and the other one differing in κ) that would apparently fit with the same experimental data. As π and κ have opposite effects, two Hill curves; in which the increase in π is compensated by the decrease in κ, and vice versa, may seem to represent the same curve, which could make it difficult to determine whether two Hill curves are different because a change in π or in κ.

Detailed analysis of the Hill curves indicates that π and κ do not compensate very well. Although curves differing in compensatory values of either π or κ may vary close each other, they do not fit exactly in any of the two regions of highest curvature (before and after the inflection point). This dispersion is caused by the fact that π, but not κ, changes the slope at the inflection point of the Hill curve. Therefore, ε, which is the slope of the Hill curve at the inflection point, can be used to easily differentiate between two Hill curves that apparently compensate for π and κ.

Conclusions

The application of the Hill analysis to resolve complex biological processes is effective for the precise and objective understanding of processes like virus or vaccine action, entry, genome replication, transgene expression, vector/transgene immunogenicity, cytotoxicity and other such parameters. The analysis is independent of the virus vaccine, vector and protein type involved and from the output parameter and variable measured, such as the internalized vector DNA, transgene mRNA level and transgene product activity.

As in the field of chemical pharmaceuticals, the structure of the potential drug (in this case the biological agent) must be optimized to a maximal possible intrinsic potency. In analytical development, the goal is to search for better performing reporter systems (the lowest possible K), as analytical tool. Two different systems characterized by constants KA and KB, respectively, can be compared (using the same biological agent) for their relative resistance or performance.

Complex systems involving the interaction of biological agents, such as viruses, vaccines, gene transfer vectors, antibodies proteins and living cells (either in vitro or in vivo) can be analyzed using the Hill equation. A complex succession of unitary processes, each of them susceptible to be individually analyzed by the Hill equation, as a global process, can be also described by the same equation as its constitutive steps.

Since modifications will be apparent to those of skill in this art, it is intended that this invention be limited only by the scope of the appended claims.

What is claimed is:

1. A method for the production of a molecule, having a predetermined property, comprising:
(a) producing a population of sets of target functional nucleic acid molecules that each comprise a target modified functional sequence of nucleotides;
(b) introducing each set of nucleic acid molecules into host cells and expressing a protein whose expression is modulated or regulated by the target functional sequence of nucleotides, wherein the host cells are present in an addressable collection; and
(c) individually screening the sets of encoded proteins to identify the target functional nucleic acid molecules whose activity is altered, wherein each such target functional nucleic acid is designated a hit.

2. The method of claim 1, wherein the target functional sequences of nucleotides is selected from the group consisting of cis acting regulatory sequences, antisense nucleic acid and RNA.

3. The method of claim 1, wherein the target functional nucleic acid is selected from the group consisting of promoters, enhancers, silencers, protein binding domains, ribosomal RNA and ribozymes and tRNA and antisense molecules.

4. A method for the production of a functional nucleic acid molecule having a predetermined property, comprising:
(a) producing a population of sets of modified nucleic acid molecules that encode modified forms of a target functional nucleic acid, wherein each modified functional nucleic acid molecule is operably associated with a nucleic acid region encoding a reporter;
(b) introducing each set of nucleic acid molecules into host cells under conditions that express a reporter when using a wild-type functional nucleic acid region, wherein the host cells are present in an addressable collection;
(c) individually screening the sets of nucleic acid molecules encoding reporter proteins to identify one or more target modified functional nucleic acid regions that has activity that differs from the unmodified functional nucleic acid molecule, wherein each such molecule is designated a hit.

5. The method of claim 4, wherein each the target functional nucleic acid molecule comprises cis-acting nucleic acid region.

6. The method of claim 5, wherein each cis-acting nucleic acid region comprises a promoter region.

7. The method of claim 4, wherein the target functional nucleic acid is selected from the group consisting of promoters, enhancers, silencers, protein binding domains, ribosomal RNA and ribozymes and tRNA and antisense molecules.

8. The method of claim 4, wherein the reporter is a detectable protein.

9. The method of claim 8, wherein the reporter is a fluorescent protein.

10. The method of claim 4, wherein the reporter is a detectable nucleic acid molecule.

11. The method of any of claims 1-3, wherein each target modified functional nucleic acid molecule differs by a single base or base pair from the target molecule.

12. The method of claim 4, wherein each polynucleotide in a set differs by one base from the unmodified nucleic acid molecule.

13. The method of any of claim 1 or claim 4, wherein the modified nucleic collection comprises a solid support with wells; and each well contains one set of cells.

14. The method of claim 1 or claim 4, wherein the nucleic acid molecules comprise viral vectors; and the cells are eukaryotic cells that are transduced with the vectors.

15. The method of claim 14, wherein the viral vector is selected from the group consisting of vectors derived from herpes simplex virus (HSV), pseudorabies virus and other herpes virus vectors, MuMLV, MoMLV, feline leukemia virus, and HIV and other lentiviruses.

16. The method of claim 14, wherein the viral vector is selected from the group consisting of vectors derived from adenoviruses, adeno-associated viruses, herpes viruses, vaccinia viruses and retroviruses.

17. The method of claim 1 or claim 4, wherein the nucleic acid molecules comprise plasmids and the cells are bacterial cells.

18. The method of any of claim 1 or claim 4, further comprising:
(d) modifying the nucleic acid molecules that comprise the hits, to produce a set of modified hits;
(e) introducing each set of modified hits into cells; and
(f) individually screening the sets of cells that comprise the modified hits to identify one or more cells that contain a candidate functional nucleic acid has activity that differs from the target functional nucleic acid and has properties that differ from the original hits, wherein each such functional nucleic acid is designated a lead.

19. The method of claim 4, wherein the nucleic acid molecules in step (a) are produced by a method selected from among nucleic acid shuffling, recombination, site-directed or random mutagenesis.

20. The method of claim 18, wherein the nucleic acid molecules in step (d) are produced by a method selected from among nucleic acid shuffling, recombination, site-directed or random mutagenesis, and de novo synthesis.

21. The method of claim 1 or claim 4, wherein the modified target functional nucleic acid molecules in step (a) are produced by systematically changing each base in the target protein to a pre-selected base.

22. The method of claim 18, wherein the nucleic acids of step (d) are produced by systematically replacing a base that is a hit, with each of the remaining two bases, to produce nucleic acid molecules each differing by at least one base each designated as modified hits to identify leads.

23. The method of claim 22, further comprising:
recombining the nucleic acid molecules that are leads;
introducing those nucleic acid molecules into cells; and
screening the cells to identify nucleic acid molecules that comprise optimized leads.

24. The method of claim 23, wherein the recombining is two, three or more up to all of the nucleic acids comprising the leads.

25. The method of claim 24, wherein the recombining is effected by a method selected from among nucleic acid shuffling, recombination, site-directed or random mutagenesis and de novo synthesis.

26. The method of claim 1 or claim 4, wherein the modifications are effected in a selected portion of the target functional nucleic acid molecule.

27. The method of claim 1 or claim 4, wherein the target functional nucleic acid molecule is a nucleic acid molecule that comprises a cis-acting nucleic acid region.

28. The method of claim 1 or claim 4, wherein the change in activity is at least about 0.1%, 1%, 5%, 10%, 20%, 30%, 40%, 50%, 100%, 200%, 500% or 1000%

29. The method of claim 14, wherein at step (b) the titer of the viral vectors in each set of cells is assessed.

30. The method of claim 15, wherein at step (b) the titer of the viral vectors in each set of cells is assessed.

31. The method of claim 1 or claim 4, wherein the nucleic acid molecule comprises a promoter and the initial sequence is gradually converted by mutagenesis into corresponding consensus sequences known to have higher activity by accumulation of single point mutations from the borders towards the middle of the consensus sequences to generate a collection of mutants, wherein each mutant differs from the preceding mutant in that a new mutation is added.

32. The method of claim 1 or claim 4, wherein the nucleic acid molecule comprises a promoter and each of the single base deletion mutants is generated such that each mutant has either increased or decreased distance between one or more bases in the consensus sequences in the promoter by virtue of insertions or deletions.

33. The method of claim 31, wherein the nucleic acid molecules that exhibit the desired activity are selected.

34. The method of claim 33, wherein the desired activity is an increased production of a protein encoded by a sequence of nucleotides operatively linked to the promoter region.
